(19) United States
(12) Patent Application Publication
(10) Pub. No.: US 2011/0066083 A1
(43) Pub. Date: Mar. 17, 2011

(54) Ultrasonic apparatus and method for treating obesity or fat-deposits or for delivering cosmetic or other bodily therapy

(76) Inventors: Carol A. Tosaya, Los Altos, CA (US); Lee Blumenfeld, Lincolnshire, IL (US); John W. Sliwa, JR., Los Altos, CA (US)

(21) Appl. No.: 12/807,965

(22) Filed: Sep. 17, 2010

Related U.S. Application Data
(62) Division of application No. 11/262,452, filed on Oct. 27, 2005.
(60) Provisional application No. 60/623,535, filed on Oct. 28, 2004.

(51) Int. Cl. A61N 7/00

(52) U.S. Cl. 601/2

(57) ABSTRACT

A wearable treatment or therapy apparatus for provision of an acoustically enabled or acoustically enhanced treatment or therapy to a patient or treatment subject is provided. Also provided are apparatuses for delivering an acoustic or acoustically-aided therapy or treatment to a patient or treatment-subject.

### CROSS-REFERENCE TO RELATED APPLICATION

The present application is a divisional application of application Ser. No. 11/262,452, filed Oct. 27, 2005, the contents of which are incorporated herein by reference, which in turn claims priority from provisional application Ser. No. 60/623,535, filed Oct. 28, 2004.

### BACKGROUND OF THE INVENTION

1. Field of the Invention

The present invention is directed to treating obesity or fat deposits, and, more particularly, to the use of ultrasound in such treatment.

2. Description of Related Art

A. Introduction

Ultrasonic treatment of mammalian obesity, fat-deposits, cosmetic issues or for delivering bodily-therapy is not a new idea. Fat deposits indicative of obesity are widely known to contribute to a number of debilitating or life-threatening diseases such as diabetes and heart disease. Numerous inventors have patented a wide variety of vibratory, sonic and ultrasonic means, some of which are claimed to operate effectively in conjunction with particular recommended drugs and skin-applied medications to reduce fat or ameliorate visible imperfections such as cellulite. Others claim to beneficially treat muscles, visceral tissues or organs, improve circulation or accelerate wound, burn or injury-healing. Very few of these inventors have provided clinical proof of the workability of these methods, apparatus and compositions and only a handful have attracted serious investment funding despite having little or no clinical evidence of workability in humans, unproven markets and unknown, questionable and/or unclear need for regulatory acceptance. However, we have recently begun to hear of some limited clinical and lab-results for obesity or cosmetic fat treatment which appear to have some scientific basis. Utilizing these recent results we herein provide a variety of new apparatus and methods for obesity and unrelated acoustic procedures which implement currently understood therapy mechanisms as well as future anticipated mechanisms in a manner which offers the user, for the first time, complete safety of operation and uniformity of treatment. The inventive apparatus and associated methods, as far as we are aware, are also the first which can offer a home-use embodiment as well as a wearable embodiment that is safe both from the potential shock-hazards but also from the potential over-treatment hazards.

B. The Prior Art

Regarding the prior art we shall focus on the most demanding therapy applications requiring the most of such apparatus. The treatment of fat and cellulite is probably the most challenging because one either destroys or degrades cells or at least encourages the body itself to destroy or burn fat. Fat cells or adipocytes are known or thought to be degraded and/or destroyed by ultrasound, directly or indirectly, via several different mechanisms. The challenge is to perform such ultrasonic therapy without causing other injuries such as hemolysis, tissue burns, nerve-damage or organ-damage. The relevant prior art involving ultrasound, vibration or sonics for fat, cellulite and cosmetic treatment can be divided up into a few mechanism or mechanistic categories as follows:
1. Ultrasonic therapies, treatments or surgeries which thermally damage adipocytes (fat cells) or critical parts thereof, directly or indirectly;
2. Ultrasonic therapies, treatments or surgeries which cavitationally damage adipocytes or critical parts thereof, directly or indirectly;
3. Ultrasonic therapies, treatments or surgeries which promote or encourage the release or activation of in-situ generated lipolyzing agents, directly or indirectly; and
4. Other therapy, treatment and surgical methods and apparatus

We shall now provide examples of each of these categories reminding the reader that our own invention herein may utilize one or more of these mechanisms as well as future-discovered mechanisms.

B1. Thermally Damaging Adipocytes.

Let us begin by saying that the delivery of heat to tissues via ultrasound heating for beneficial medical purposes is not at all new. There has been a history of work in the area of hyperthermia wherein electromagnetic (RF and microwave) or ultrasound-induced heating of tissues either accelerates the action of an anti-cancer drug or the mild heating is used to directly kill metastatic cancer or infections such as HIV. Hyperthermia typically heats the tissues modestly, a few degrees C., such that healthy cells can survive. Hyperthermia is quite distinct from HIFU (high-intensity focused ultrasound) wherein a highly focused transducer burns or necroses a tumor or fat by heating it several tens of degrees C. Such HIFU therapy usually kills all cells in the acoustic focus region so HIFU must be aimed very carefully. In any event these are all implementing at least thermal damage.

Others investigators have used ultrasound-induced heating to specifically melt or dissolve fat. U.S. Pat. No. 4,886,491 to Parisi teaches an invasive liposuction probe with ultrasonic 40 khz excitation and the use of infused saline. The ultrasonic energy, via fat-cavitation for example, heats, melts and/or emulsifies the fat and saline such that it can be sucked out of the hollow tubular ultrasonic liposuction probe. U.S. Pat. No. 5,143,063 to Fellner teaches the use of externally noninvasively focused ultrasound, RF or microwave energy to thermally destroy adipose tissue or fat via thermal necrosis. Finally, WO00132091A2 to Riaziat also describes a means of noninvasively thermally necrosing fat using ultrasound or RF energy in combination with a surface cooling plate to prevent superficial skin from burning. We note that if one is cavitating strongly in fat then one is also producing at least some modest heat due to the cavitation itself and the power-level required to cavitate. It is widely known that cavitation bubbles attain super-hot temperatures of over a thousand degrees K at least for very short moments at least within their volume. We also note that ultrasonic frequencies most useful for producing such cavitation, namely low frequencies in the general range of tens of kilohertz up to a megahertz or so, do not cause high heating of tissues without such cavitation due to their inherent low attenuation. Thus the reader will realize that a fat cavitation treatment generates at least modest heat, at least locally at the cavitation bubbles, and it is therefore not completely true that such a cavitation treatment is a completely “nonthermal” treatment. In order to keep a cavitation method from also becoming a thermal method one usually utilizes short ultrasonic pulses and low duty-cycles allowing for cooling between cavitation events. If one utilized sufficiently long continuous waves of 100% duty-cycle to cavitate, one would also experience significant heating-even at the weakly attenuating low frequencies mentioned.

Liposonix of Bothell, Wash. is a startup company proposing to use ultrasound to perform noninvasive body-sculpting. Because the procedure is one of several new noninvasive ones, the fat that is liberated (or destroyed) within the body needs to be processed by the body itself as opposed to being sucked out as by liposuction. Such totally noninvasive products and methods are intended to make a dent in the conventional invasive liposuction market. US2004039312 A1 to Hillstead et al of Liposonix describes a noninvasive ultrasonic lipolysis system having boiling and cavitation sensors. The patent application says little or nothing about recommended operating conditions—only that the two mentioned sensors may be used to control the therapy process. Thus a therapy that was entirely mainly thermal or mainly cavitational or both of these could be monitored or controlled by one or both such sensors. Thus we will include this apparatus under both thermal tools and cavitational mechanisms. It will be noted that the Liposonix inventive device has a complex tracking and/or guidance system to assure uniform treatment and assure no over-treatment. WO03070105A1 to Cribbs of Liposonix is equally vague about their recommended operational parameters-however there is some industry-journal indication that normal operation will involve both cavitation and boiling mechanisms.

B2. Cavitationally Damaging Adipocytes.

There are numerous patents pertaining to using cavitation to treat a variety of pathological or undesired tissues without necessarily specifically focusing on adipose tissues-but seemingly covering adipose tissues. Among these are U.S. Pat. No. 5,143,073 to Dory, U.S. Pat. No. 5,209,221 to Reidlinger, U.S. Pat. No. 5,219,401 to Cathignol, U.S. Pat. No. 5,301,660 to Rattner which may employ cavitation, U.S. Pat. No. 5,419,761 to Narayanan (invasive), and U.S. Pat. No. 6,607,498 to Eshel at Ultrashape. The noninvasive fat-attacking Ultrashape™ device clearly operates by relatively low-frequency cavitation in a mainly nonthermal manner using short pulses. Thus its thermal destructive component is probably nil. US2003083536A1 also to Eshel and Ultrashape Inc. is similar to U.S. Pat. No. 6,607,498 in terms of the intentional cavitation being done in a mainly nonthermal pulsed manner. We note here that both the Liposonix device and the Ultrashape device are noninvasive and rely on the human body to absorb or process the released fat cells or fat-fragments. We also remind the reader that the Liposonix device also appears to be able to operate with cavitation. Reliance on the body to naturally dispose of destroyed fat cells is not yet been proven safe but will likely be in the future, at least for small quantities of such fat byproducts being released in an exercising patient over an extended or multisession period.

B3. Ultrasonic Therapies which Promote or Encourage the Release or Activation of In-Situ Generated Lipolyzing Agents.

It turns out that even relatively low-power ultrasound can cause chemical or physical changes in tissue which can contribute to adipocyte degradation or destruction. Several examples of these approaches are now mentioned. First we have U.S. Pat. No. 5,507,790 to Weiss which teaches heating adipose tissue tens of minutes to a temperature range of 40-41.5 Deg C. which nondestructively thermally accelerates the body's own lipolysis processes. Second, we have EP01060728A1 and WO09853787A1 to Miwa which admirably teach the use of low-power and diagnostic power-level ultrasound exposures to biochemically excite the lipolysis process as by encouraging the release of natural lipolysis hormones or by disrupting the adipocytes own phospholipids layer. Both of these patents are of great interest because of the low-power ultrasound or lack of ultrasound utilized.

B4. Other Mechanisms.

Cooling damage (as opposed to heating damage) has been directed at adipocytes and their contents. WO03078596A2 to Anderson teaches the selective disruption of lipid-rich cells by cooling. Taught therein is that cells having less lipid-rich contents, such as skin cells, are less susceptible to such cold damage than are lipid-rich cells such as adipocytes. Anderson teaches the imposition of a thermal gradient such that the adipocytes are damaged by cold temperature whereas the near-surface cells are not damaged because of their inherent greater resistance to cold. The teaching explains possible mechanisms for workability as being fat crystallization-induced ruptures of the adipocytes and/or simple thermal activation of natural lipolysis. In our own invention herein we shall also improve upon this approach.

Several patents or filings address the use of diets, skin-applied salves or ointments, skin patches, systemic drugs and even genetic molecular biology means to chemically or biologically cause fat or fat-formation disruption. We mention these because one or more of these may be used in combination with the use of our own inventive apparatus and method. In fact, several inventors have taught the driving through the skin of various drugs for various purposes by both ultrasound and various electroporation and iontophoretic means. Such drugs are occasionally mentioned therein as being anti-fat drugs or anti-cellulite drugs. Included in this drug/biotechnology list are the following patents or patent-filings: U.S. Pat. No. 5,884,631 to Silberg teaches a noninvasive ultrasonic technique using an injected tumescent fluid which is claimed to enhance ultrasonic direct destruction of adipocytes and/or the indirect destruction of adipocytes via an attack on their connective surrounding tissue. Silberg describes both invasive (suction) fat remnant removal as well as natural bodily removal. We emphasize that in the scope of our own invention herein that by “attacking fat cells” we mean directly and indirectly such as by their direct rupture or melting or as by damaging or freeing them as by attack of their membranes and/or connective tissues. U.S. Pat. No. 6,039,048 also to Silberg is similar in nature. U.S. Pat. No. 6,746,695 to Martin teaches the use of plant-extracts for a diet-based antiobesity program. US2003133961A1 to Nakamura teaches cosmetically applied gel-based compositions for an antiobesity or antifat program. US2004106123A1 to Smolyar and US2004106538A1 to Hariharan both teach genetic drug approaches to antiobesity therapy. US2004106576A1 to Jerussi and US2004106583A1 to Jaehne both teach drug-based approaches to antiobesity therapy. US2004115135A1 to Quay teaches a mucosal-delivered antiobesity drug. US2004122033 to Nargund teaches the use of combined drugs for an antiobesity therapy. US2004122038A1 to Hammond and US2004122046A1 to Elliott both teach the use of NPY-5 antagonists to suppress appetite for purposes of obesity control. US2004122091 to Dasseux teaches the use of sulfoxide compounds to treat obesity. US2004126852A1 to Stewart teaches the manipulation of fibroblast growth factor in controlling obesity. US2004127415A1 to Hsu teaches the use of stresscopins to suppress appetite and obesity. US2004127518A1 to Piomelli teaches the use of anti-anxiety drugs to treat obesity. US2004132745A1 and US2004132779A1 both to Bertinato teach the use of microsomal triglyceride transfer protein manipulation to treat obesity. US2004146908A1 to Adams also teaches the use of fibroblast growth-factor manipulation for obesity treatment. WO04045560A2 to Girouard teaches the use of monosaturated fatty-acid manipulation for the treatment of obesity, WO04047855A2 to Meise teaches the manipulation of proteins involved in the regulation of energy homeostasis in obesity treatment. WO04052864A1 to Dow teaches the use of Pyrazole and Imadazole compounds to treat obesity, WO04055002A1 also to Elliott teaches the use of Carbazole derivatives and NPY-5 antagonists to treat obesity, WO04054981A1 to Hammond also teaches the use of NPY-5 antagonists such as Aminophenanthridine derivatives to treat obesity. WO04056314A2 to Quay again teaches mucosal-delivered Y2 receptor obesity therapies. WO04056775A1 and WO04056777A1 again both to Bertinato again teach the use of microsomal triglyceride transfer-protein inhibitor manipulation to treat obesity, WO04058988A2 to Han teaches the use of binding-agents which inhibit myostatin as an obesity therapy. WO04060268A2 to Eglington teaches the use of a drug-bearing skin patch to treat cellulite. WO04062685A2 to Bloom teaches the use of OXM drugs such as for inhibiting appetite. WO04063218A1 to Collier teaches a number of obesity therapies based on manipulation of obesity genes. Some clinics are known to administer substances such as vitamins or beta-carotenes to treat obesity. Guarente of MIT has recently demonstrated that the sirt1 gene can suppress fat cell growth. Kolonin has recently demonstrated (Nature Medicine, published on-line 9 May 2004) in obese mice that injection of a chimeric peptide selectively attacks blood vessels which feed the growth and maintenance of adipose cells. The attack thereby indirectly kills the adipose cells. Finally, it has also been suggested by Kondo et al (International Journal of Radiation Biology, 1988, vol 54, No. 3 pp 475-486) and (International Journal of Radiation Biology, 1988, vol 54, No. 6, pp 955-962), particularly for cavitating processes, that cavitation-induced chemical radicals can kill cells-however it is currently postulated based on Kondo's evidence that the main cell-damage comes from the shear-stresses and microjetting or microstreaming developed by cavitation.

We have not spoken much about existing surgeries such as stomach-stapling to inhibit food intake. Obviously these are radical and can be very invasive. WO04082763A1 to Aldrich reviews some of these and teaches a new means of interrupting the vagal nerve by ablation in order to suppress appetite. This ablation is done using a transesophageal probe so it is minimally invasive surgery. While our invention herein strives to avoid very-invasive procedures we believe that there will be cases where use of our invention may be combined with a preferably minimally-invasive technique such as using the Aldrich device.

It will be noted by those familiar with the invasive liposuction art that there are numerous nonultrasonic and ultrasonic-based invasive techniques in wide use. We have only mentioned one or two of these invasive approaches above as our inventive focus below is preferably on noninvasive obesity or fat-therapies or treatments which would logically replace or displace invasive techniques. The present inventors believe that a totally noninvasive approach is preferred—and that the second less-attractive but still useful preference is for a technique wherein adipocyte destruction is done noninvasively and the fat is subsequently removed via suction through minimally invasive very small incisions or punctures. The gold standard would be a totally noninvasive technique wherein fat destruction and fat/fat byproduct removal are both noninvasive as wherein externally applied ultrasound allows for damaged fat to be absorbed or otherwise processed by the body itself in controllable amounts without complications.

### SUMMARY OF THE INVENTION

The present invention has three primary embodiments.

The first embodiment is a waveguide-based apparatus and method for applying ultrasound to a treatment-subject for the purpose of providing treatment or therapy for obesity, fat-deposits, cosmetic benefit or other bodily therapy tasks such as improved skin-properties or injury treatment. Several mechanisms by which the treatment achieves its benefit are taught and one or more of these mechanisms can be practiced using the invention.

The second embodiment is an entirely novel apparatus and method for providing at least one such treatment or therapy using a liquid-based waveguide.

The third embodiment is a wearable apparatus that incorporates a waveguide of the invention.

Any of the embodiments has application to hospital-use, clinical-use or home-use, for example, and the place of use will likely be determined by which treatment mechanism is employed and at what power-level. There are FDA and other regulatory requirements (power, intensity or temperature limitations as mentioned in the Miwa references) for such mechanisms which will be mentioned herein and are long-known to those skilled in the medical ultrasound product-development and regulatory approval areas.

Our above prior art discussion focused on known beneficial operative mechanisms in detail, while mentioning some of the specific prior art apparatus of relevancy. It is the aim of these inventive devices herein that they can be designed and operated to practice one (or possibly more) of the above mechanisms in the manners known to be of benefit from that art. So the first embodiment is an improved apparatus capable of practicing any or all of these mechanisms as well as future mechanisms for any such therapy or treatment. The second embodiment based on liquid-streamed waveguides, as mentioned, is in one variation, an entirely new apparatus and in the other also an improved apparatus. The third wearable embodiment is preferably utilized at-home or out-of a clinic and utilizes relatively low-power ultrasonic therapies not necessarily requiring regulatory approval.

### BRIEF DESCRIPTION OF THE DRAWINGS

FIG. 1a depicts a simple elongated waveguide or wave conduit guiding an energy type from left to right.

FIG. 1b depicts an apparatus employing an acoustic energy source delivering energy into an elongated waveguide and then rightward into a treatment subject without any additional focusing.

FIG. 1c depicts an apparatus employing an acoustic energy source delivering energy into an elongated waveguide and then rightward into a treatment subject with beam spreading.

FIG. 1d depicts an apparatus employing an acoustically edge-excited or end-excited vibrating plate-like. bar-like or membrane waveguide emitter.

FIG. 1e depicts an apparatus employing an acoustically surface-excited vibrating plate-like, bar-like or membrane waveguide emitter with one transducer.

FIG. 1f depicts an apparatus employing an acoustically surface-excited vibrating plate-like, bar-like or membrane waveguide emitter with multiple transducers.

FIG. 2a illustrates a bathtub or Jacuzzi® hot tub equipped with a multitransducer excited plate-like or membrane waveguide emitter.

FIG. 2b illustrates a bathtub or Jacuzzi® hot tub equipped with a multitransducer excited plate-like or membrane waveguide emitter as well as a handheld emitter.

FIG. 3a shows a therapy applicator of the apparatus delivering two shown continuous streams of liquid which act as acoustic waveguides for acoustical energy passing therethrough.

FIG. 3b shows an applicator of the apparatus delivering two shown droplet streams of liquid which act as their-own acoustic excitation means upon impact with the treatment subject due to the continuous conversion of droplet kinetic energy to pressure waves.

FIG. 3c shows a wearable therapy-delivery portion of the apparatus of the invention for strapping onto the thigh, for example.

### DETAILED DESCRIPTION OF THE INVENTION

For the invention herein we utilize the following definition of “waveguide”. Note carefully that our waveguide can have two very different generic forms:
Waveguide:
   Form 1—A body of material(s) permitting energy-propagation through or along one or more material mediums, the body accepting an energy input and delivering an energy output of at least some of that energy in a manner and form having a useful beam-pattern or intensity-field. The body of material(s) may, if desired, mode-convert, spread, redirect, focus, defocus, polarize, disperse, phase-modify, modulate, collimate, bleed, leak, or otherwise change a propagation parameter of said energy relative to an input parameter state.
   Form 2—A series of one or more moving sequentially-impacting bodies, such as impinging liquid droplets for example, which each impact a surface with an individual kinetic energy and collectively with an impact frequency, the periodic impacts acting to excite acoustic waves at or from the impact point, the string of arriving kinetic bodies effectively acting as if it were a material waveguide of Form 1 delivering acoustic waves from a material waveguide.

With regard to Form 1, it will be noted that the input and output locations, per this definition, need not be limited to one-each, limited to remote or separated locations, limited to point-locations, or even limited to being the same energy type. Even more importantly, the waveguide is not necessarily a passive energy conduit.

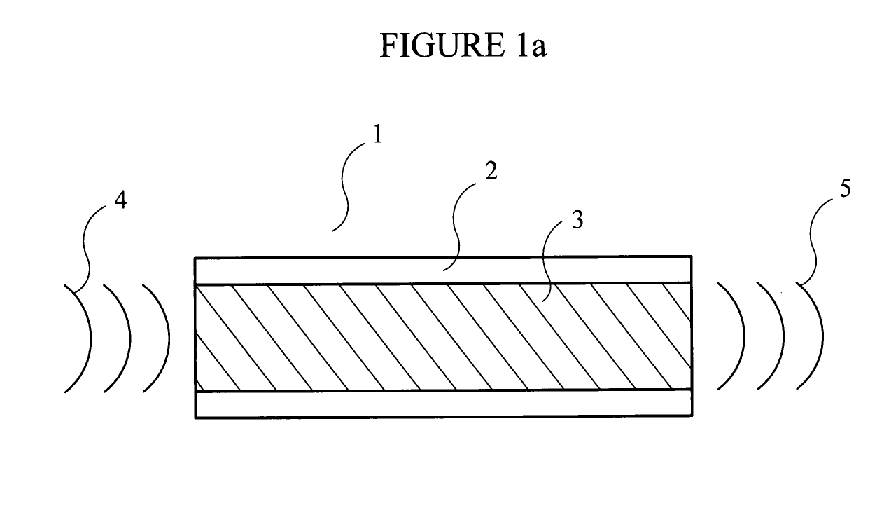

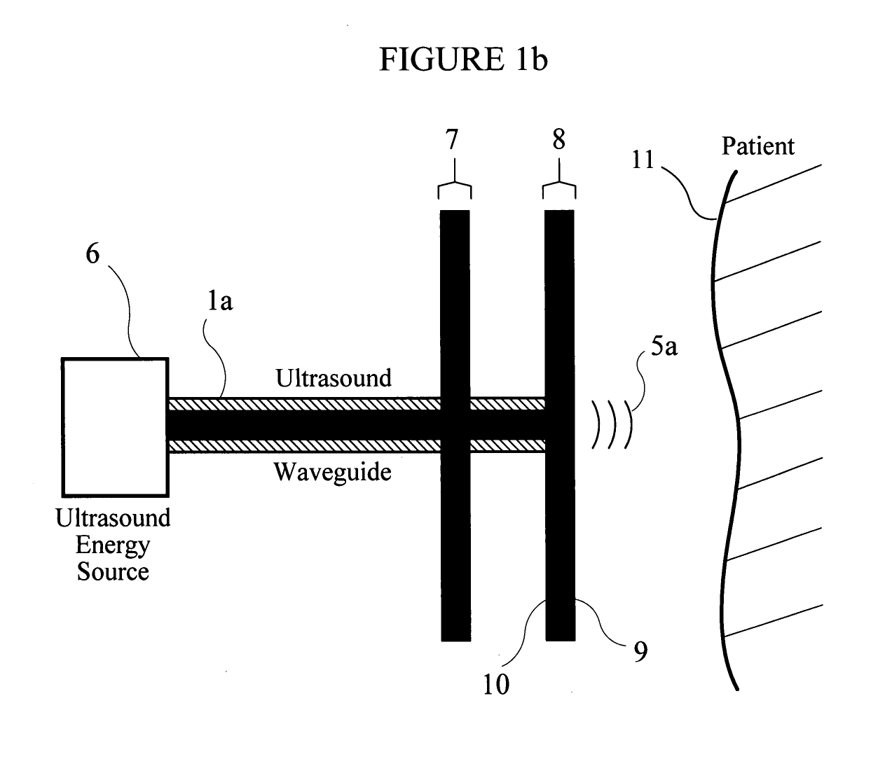

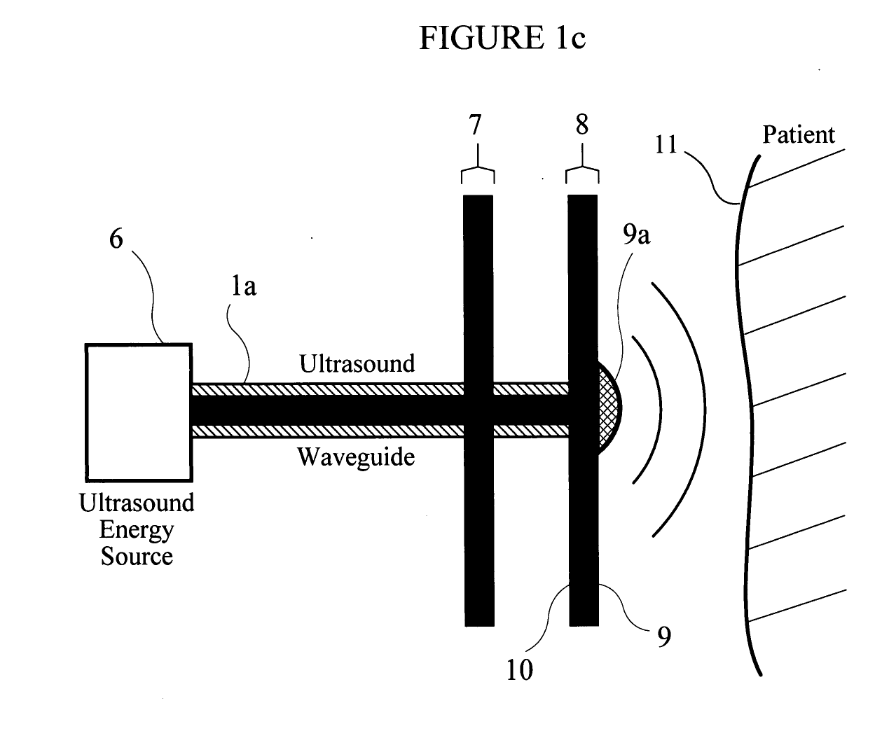

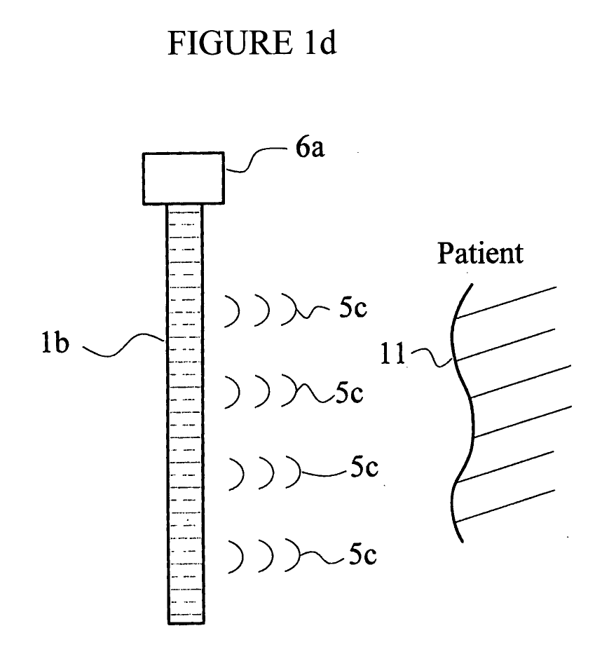

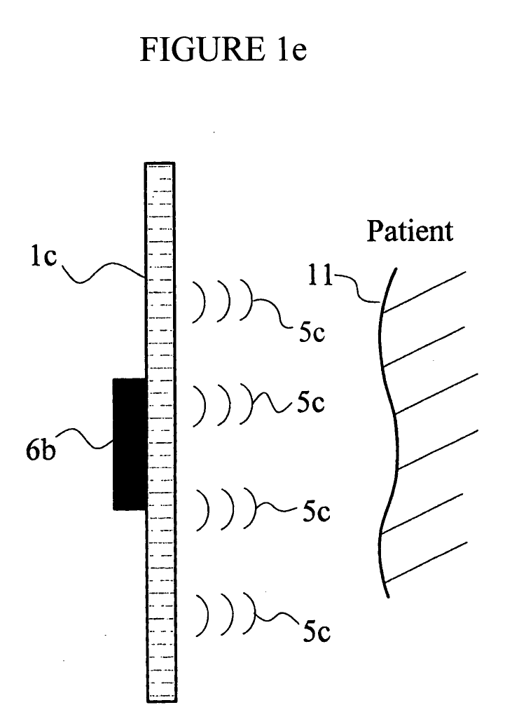

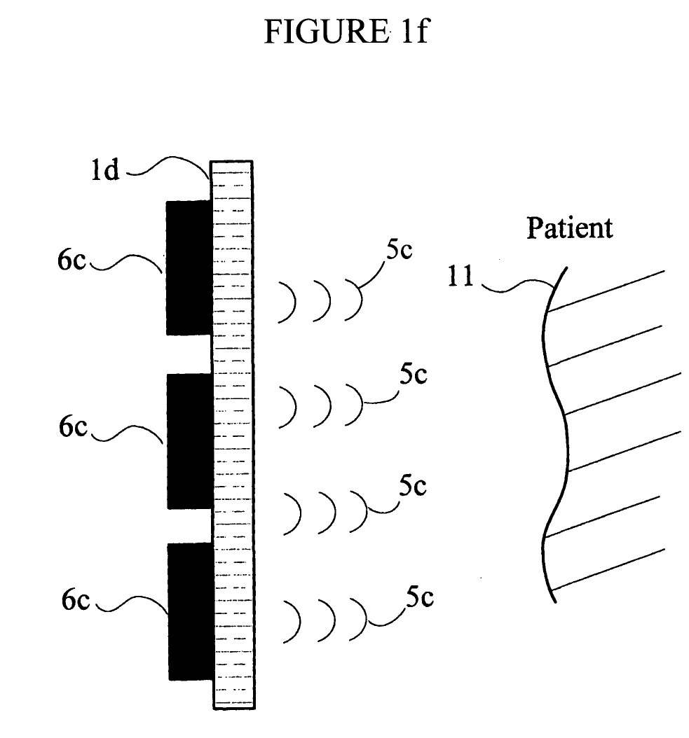

Let us begin with FIG. 1a. Therein is depicted a generic-type energy waveguide 1 having a propagation core 3 and a shell, outer sheath or cladding 2. Ingoing energy 4 is shown on the left and outgoing energy 5 is shown on the right, both of which are shown traveling to the right with little or no loss in any direction lateral to the waveguide axis. This particular waveguide of FIG. 1a is tube or rod-shaped, might be bendable (bending not shown) and its function in this example is to deliver or pass energy along its length rightward with low or at least acceptable losses such that the energy can be moved from the left end to the right end where it will be usefully utilized. A familiar type of energy-waveguide to the layman would be a fiberoptic light guide 1. In that case the energy is optical laser-energy and the waveguide has a glass-based core 3 and a different glass or polymer sheath or cladding material 2. Typically, the optical energy in that example is constrained to the core 3, because it tends to bounce off of, reflect from or refract from the interface with the sheath 2 (or from the ambient in the case where there is no sheath). Typically such guiding behavior can be engineered by choosing the core material 3 to have a different propagation property than what surrounds it—i.e., different than the sheath 2 or different than the ambient if no sheath exists. In the case of a fiber-optic, the propagation property which is different and causes beneficial energy redirection inwards is typically an optical index of refraction.

Many types of energy can be guided by an appropriately designed waveguide 1. Acoustic energy is another wave-type energy which can be guided and microwave energy as used in radar or tissue-ablation/heating could possibly be another. Further, we have defined waveguides more broadly that conventional ones (like that of fiber-optic FIG. 1 example) to include waveguides which may beneficially alter an energy parameter as opposed to passively carrying the energy with no intended parameter change. Our invention herein makes considerable use of both types of behavior.

Practitioners of the acoustic arts will be aware that acoustic waveguides, particularly of the passive point-to-point delivery type, are commonly employed. Frequently, for example, a metallic rod or wire is excited on one end and the acoustic energy delivered to the remote end of the waveguide by such guiding action. A good example of this is an ultrasonic wire-bonder bonding-tip used in microelectronic packaging wire-bonders wherein the bonding-wire is acoustically (thermomechanically) welded to a substrate using a tapered acoustic waveguide bonding-tool tip sometimes called a “horn” or capillary. The tip's taper acts to beneficially amplify the lateral microscopic scrubbing and welding vibrations. Note that for this example that the acoustic waves are primarily transverse to the long capillary axis yet they still propagate along the length axis. Another example would be the products of Omnisonics™ of Wilmington, Mass. Their acoustically excited wire-type waveguides are used for unblocking a patient's bodily vasculature as they are fed through such blocked vessels and the ultrasonic vibrations break up the blockages. Omnisonic's technology for such products is described in Patents #'s WO04058131A2, WO04012599A1, WO02070158A1, WO00213678A2, US24176686A1, US24162571A1, US24158150A1, US24171981A1, US24031308A1, US24019266A1 and US23236539A1, for example. Note that Omnisonic's waveguides typically have a single energy input at an end but have several energy output positions at antinodes along the length of the excited catheter-like wire. So Omnisonics represents a case of a waveguide driven to operate such that transverse acoustic input energy passing along its length leaks out at a number of transversely vibrating antinodes into surrounding materials. The leaked energy is a combination of pressure waves and shearwaves. Thus in just these couple of examples we already see that acoustic waveguides can not only guide energy along their lengths but can also be used to deliver or distribute energy to their ends and/or sides in a controlled fashion. The basic waveguide of FIG. 1a could be, for example, a very simple longitudinal acoustic waveguide whose job it is to deliver acoustic energy from the input 4 to the output 5 with minimal loss such that at the output 5 the still forward-traveling compressional waves can be used to perform a useful function. Note that in FIG. 1a as depicted, the physical vibrations or oscillatory displacements associated therewith are shown arranged generally parallel to the axis of the tubular waveguide 1 and comprise axial compressional and rarefactional waves and very minimal energy therefore leaks sideways or transversely to the transverse direction of left-to-right propagation. Thus waveguide 1 of FIG. 1a is shown as having desirably longitudinal waves being propagated therethrough. The wire-bonder and Omnisonics examples are similar in nature however in those the acoustic energy, rather than being primarily longitudinal compressional/rarefactional waves with physical displacements along the propagation longitudinal axis, instead have excitation displacements which are primarily transverse to the waveguide but still also propagate as waves along the longitudinal lengths. These are frequently referred to as transverse or shearing waves. Those familiar with waveguides know that, for example, a taut wire can be made to oscillate along its length (like FIG. 1a), or transverse to its length (not shown in FIG. 1a) like the Omnisonics products, or even torsionally (not shown in FIG. 1) as by a vibratory torsional driving forces. In fact, any or all of these modes can be driven separately or together. The reader will also likely be aware that virtually any physical body, such as a wire, a bar, a rod, a plate, a shell or a membrane has preferred natural (and unnatural) vibrational modes which can be selectively or simultaneously driven depending on whether the driving-oscillation encompasses or is close to those modes. The modes and frequencies thereof, such as those of a musical drumhead, are a function of the body's shape, thickness, elastic stiffness and density. In the further figures, unless otherwise noted, depicted acoustic waves such as waves 5 are traveling in an acoustic medium such as water until they enter another member or enter the body. The use of water and other acoustically propagating low-attenuation mediums is widely practiced and known to be convenient if an object emitting acoustic energy is to be coupled to a target object without direct contact.

So we see that the waveguide of FIG. 1a at least acts to control a direction or distribution of a usefully propagating wave. In fact a well-designed passive waveguide such as waveguide 1 of FIG. 1a will also act to maintain a desired mode(s) at the output 5. We depicted a compressional longitudinal acoustic wave propagating with nil lateral losses. Using our waveguide definition we can also see that the wire-bonder example represents an example of distributing and controlling ultrasonic transverse vibrational energy for use at the bonding-tip. In fact wire-bonder tips for ultrasonic bonding are frequently tapered toward the tip such that vibrational amplitude amplification takes place. This qualifies as modulation in our definition. Finally, the Omnisonics™ transversely vibrating wires are also examples of controlling and distributing transverse acoustic vibrational energy except in that example one drives the wire into transverse oscillations at a frequency such that the wire has several nodes and antinodes—and when the wire touches the bodily lumen, vasculature, blood or thrombus the antinodes deliver energy laterally into the tissue for anatomical cleaning and unblocking purposes. Note that the wirebonder tip waveguide delivered its energy from one end to the other whereas the Omnisonics transverse wire purposely bleeds off some or all of the energy before it propagates to the far end—or at least it is not primarily delivered from the end.

Being consistent with our definition of waveguide we include acoustic lenses which act to distribute (steer or direct) acoustic waves as well as acoustic matching-layers which act to enhance the passage of such waves. Finally, an acoustic mirror, reflector or refractor would also be an example of a waveguide as we define it as it redirects such waves. So, in summary, the reader should see that the “guide” aspect of waveguide means herein that the wave may beneficially be acted upon in one of several possible manners beyond simple passive propagation such that the modified, modulated or redirected propagating energy or waves can perform a useful function in a superior manner.

Moving now to FIG. 1b we see a schematic representation of a therapy or treatment apparatus of the invention. Therein we see a familiar acoustic waveguide la designed to primarily passively transport acoustic energy from left to right. On the left hand side we see an ultrasound energy source 6 which, for example, could be a piezoelectric acoustic transducer which is electrically excited. We note that transducer 6 is acoustically coupled into waveguide 1a such that emanated acoustical energy can propagate from transducer 6 into waveguide 1a. We note that on the right hand side we see the tissues of a treatment subject or patient 11 and that acoustical energy is emanating from the waveguide 1a in the form of acoustic waves 5a which are shown to be directed primarily rightwards to patient tissues 11 in a water ambient, for example. Also in FIG. 1b we also see two crosshatched plates, walls or boundaries 7 and 8. As an example the apparatus of FIG. 1b could be designed to treat a patient in a water-tub or bathtub wherein the water acts as an acoustic couplant to patient 11. So wall or bulkhead, in that example, could be the tub wall 8. Further, wall or bulkhead 7 could be a wall of the room in which the apparatus is situated. Thus we have energy waveguide 1a passing acoustic energy from a remote location, for example a utility closet, through a wall 7 and coupled into, onto or through (onto shown) the bathtub bulkhead 8 whose right hand side has a depth of water (not shown) and an immersed patient 11.

So the bathtub wall 8 whose outside surface 10 and inside (wet) surface 9 is shown passing the acoustical energy through its thickness, a thickness shown as being generally uniform as for an acoustic matching layer or an acoustic window, is simply a short intermediate path for the acoustical energy 5a. So in FIG. 1b energy 5a simply passes onto patient 11 without any further shown focusing or redirection. In other words the bathtub wall 8 simply acts as an acoustic window through which acoustics pass with little modification. As an example the tub wall 8 could be fabricated of urethane or a rubber, known reasonable acoustic window materials. Alternatively (not shown) waveguide 1a could have penetrated wall 8 in a leakproof manner-allowing for material 8 to be comprised of a material which is not a good acoustic window.

Within the scope of the invention is performing additional waveguide (see definition) related functions to acoustic energy 5a before it gets to the patient 11. Although not shown in FIG. 1b, for example, one could perform one last defocusing, focusing, collimating or acoustic-matching function to further beneficially steer, distribute, homogenize or direct the acoustical energy before it impacts the treatment subject 11. Thus plate or bathtub sidewall 8, for example, could alternatively be or could contain an acoustic lens, acoustic mirror, an acoustic collimator or an acoustic matching layer for example.

It should be clear from FIG. 1b that what we have achieved here are two things: a) acoustical treatment energy has been delivered to a patient 11 through a water-bath in a manner such that there is no electrical shock-hazard to the patient as would be the case if the transducer 6 were itself wetted or immersed in the bathtub 8, and b) we could have easily provided a second waveguide element (not shown) such as an acoustic lens, collimator or matching layer somewhere on or between one or both of bathtub walls 10 and 9 such that the patient-bound acoustic energy is, for example, redistributed with a controlled beam shape.

We note specifically that we have shown in FIG. 1b one transducer 6 and one waveguide 1a delivering acoustical energy in a primarily longitudinal mode through a point 10 into a bathtub for example. The invention has no limit on the number of exciters 6 nor waveguides 1a. Further, the invention may utilize longitudinal waves (shown) or transverse waves (not shown) or torsional waved (not shown) or any combination of these simultaneously or sequentially. In the case of, for example, transverse waves one would, as in the Omnisonics prior art, arrange the waveguide 1a or its applicator means to laterally deliver energy in the known manner.

The ultrasound energy source 6 may take any form including all manner of piezoelectric, electromagnetic, electrostrictive, electrostatic, electroscopic, magnetic, magnetostrictive and photoacoustic transduction means. Pneumatic and hydraulic exciters 6 are also known to the art as are rotating or oscillating vibration mechanisms 6. In any case, in the FIG. 1b apparatus we have the exciter 6 at least located outboard of the tub 8 such that there is no electrical or other user-hazard from the energy transduction means itself. In the extreme case the first waveguide 1a disappears and we have only the second inventive waveguide means associated with member 8 at the tub wall with the exciter mounted directly to it but outboard of it. Such an extreme case would comprise, for example, a tub or bathtub having transduction means mounted nearby behind or outboard of waterproof or water-blocking panels or membranes 9 which directly face and are wetted by the tub water on their inside surfaces. In such an approach one no longer has a long passive waveguide as shown in FIG. 1b.

FIG. 1c is very similar to that of FIG. 1b except that we have schematically depicted that member 8 now is or has incorporated in it or on it a defocusing or energy-steering means or lens 9a such that acoustical energy 5b is distributed upon the patient 11 more widely. Again, we stress that if, for example, means 9a were an acoustic lens or defocusing means as shown then it may be formed or molded as part of the tub wall 8 or may be provided (as shown) as a component situated on or in the tub wall 8. In any case, the assemblage 8/9a will be arranged so as not to undesirably leak water or other utilized liquid from right to left. Again, in FIG. 1c, wall 7 might be for example, a room wall such that excitation means 6 is remote and/or hidden to provide silence or further shock-risk reduction. Alternatively, in FIG. 1c, there might be no waveguide-penetrated wall 7 and the first waveguide 1a has minimal or zero length, and the excitation means 6 is mounted directly on an outer surface of the tub wall 8 and the acoustical energy is delivered across tub wall or membrane 8 with the help of a cointegrated or attached 9a waveguide lens 9a. In any event, there will always be at least one waveguide, per our definition herein, as part of the apparatus and in many cases there will be two waveguides, one of the general passive type 1a to deliver energy from a remote or distal transduction means and one such as 9a to steer (and/or achieve better acoustic matching) into the tub water.

Let us now move to FIG. 1d. Therein we see an example of an edge or end-excited bar, rod, plate or membrane waveguide 1b in side-view. For the sake of useful example it is a membrane 1b, A membrane is a sort of flexible or flexural plate-possibly, but not necessarily, being elastically tensioned in its own plane such as if it were of elastic polymeric material. It can also be a flexurally distortable metal or ceramic plate which is itself self-supporting. Transducer 6a of FIG. 1d is shown exciting lateral waves in the plate 1b such that several transverse vibrational nodes and antinodes are set up. We show acoustical energy 5c being leaked off or bled off into the water which couples waves 5c to the patient 11. Four such antinodes result is four locations of energy bleedoff laterally as depicted. Assuming item 1b is a flexural two-dimensional plate (flexural meaning it at least will microscopically flex transversely when acoustically driven by transducer 6a) it will be recognized that the shown antinodes (and nodes) are extended into the paper assuming the driving exciter 6a is also extended in that manner. Thus we have four lines of acoustic excitation being directed sideways toward the patient 11. By varying the excitation frequency one can excite various numbers of nodes and antinodes in different positions, for example 3 antinodes, then 4, then 5, etc., thus covering a varying swath(s) of tissue 11. Other vibrational modes may also be excited by having multiple such transducers and by having the flexural plate have a nonuniform thickness, shape, mounted masses or stiffeners, or pattern of holes in it. In any event flexural plate or membrane 1b is a waveguide per our definition and is highly useful to distribute or spread acoustical energy to patient 11. As before, plate or membrane 1b may be located outside a tub-wall for example, or may be part of the tub-wall. In any event our invention preferably does not place an electrically operating transducer in the water in which the patient is immersed. If we were to have a waveguide plate or membrane such as 1b in the tub itself, such as part of an applicator, we would preferably have it connected to the outside tub environs via an acoustic conduit of the general type 1a. Means may also be provided to mechanically scan a waveguide plate or bar 6a via translation or rotation for example, such that a larger volume or area of the patient is addressable. Such implementations are in the scope. We also include in the scope the idea of moving an excitation transducer 6a to different plate or membrane 1b positions (or even different plates) to further vary the possible excitation repertoire. Finally, acousticians will know that even a single localized point transducer 6a mounted on an edge or corner or middle of a large plate or membrane 1b can excite complex two-dimensional displacement patterns depending on frequency and a variety of plate parameters including stiffnesses, masses and inertias. This is similar to the widely known Bessel Functions which mathematically predict the displacement patterns of the drumhead when struck in various locations.

Moving now to FIG. 1e we see a somewhat similar arrangement as that of FIG. 1d except in FIG. 1e we have a back-excited plate or membrane 1c. A transducer 6b is shown laminated or otherwise acoustically attached to the plate or membrane 1c left face. Depending on the mode of operation of the transducer 6b with respect to frequency and/or bandwidth one can excite a variety of resonances and nonresonant vibrational states in the plate or membrane 1c. As an example a round plate or drumhead 1c can be excited into the many modes described by the well-known Bessel Functions. In FIG. 1e the transducer 6b may be arranged to operate in one or more modes, as is true for all of our waveguides, depending on which mode(s) the excitation transducer is driven in. For example, two widely known modes are the compressional mode and the shear mode. Operating in more than one mode simultaneously or sequentially will further enhance the variety of vibrational states into which the waveguide plate or membrane 1c can be driven. Those familiar with vibration realize that most mechanical systems such as these can be most efficiently be driven on a resonance such as a lower-order mode. We do not constrain the invention to on-resonance operation despite the likely poorer efficiency off-resonance. Since our applications sometimes have a body of water nearby, such as in the tub examples, we anticipate that significant heat can be sunk from the transducer into the tub water (or tub wall) if desired despite it operating in less than an optimally efficient vibration state. Further, we include in the scope the use of varying frequency, scanned frequency, and chirped frequency as well as broadband wide-frequency operation and duty-cycled on/off operation. We note that recently we have seen a number of companies making audio speakers that look something like the apparatus of FIG. 1e. By varying the frequency and having two or more transducers one can produce a wide range of audio speaker-like sounds from such membrane speakers. Not that those devices are differently air-coupled and of very low frequency. Water or acoustic-couplant is also avoided just as one would not let stereo speakers get wet.

Moving now to FIG. 1f we have a similar waveguide plate or membrane 1d but this time waveguide plate or membrane 1d is excited by three back-mounted transducers 6c. Such transducers may be operated in synchrony or may be operated in the well-known phased-array manner with time delays between them. In an event, particularly if each of the multiple transducers 6c is separately electrically addressable (fireable) one can deliver pulsed or CW phased (or not phased) excitations to urge waveguide plate or membrane 1d into a wide variety of vibrational resonant and/or nonresonant vibrations or vibration modes. Again in FIG. 1f we see four antinodes delivering acoustical energy 5c to the patient 11. Again we would preferably arrange transducers 6c to be outside any patient-immersion water but that could be done, for example, by having waveguide plate or membrane 1d be part of the wall of the bathtub itself for example. Again, rather than mounting three transducers upon waveguide plate or membrane 1d one could alternatively attach three waveguides of the general type 1a so that waveguide plate or membrane 1d could then be used in an applicator within a tub safely, as on he end of a flexible acoustic conduit or waveguide 1a. We note specifically that in the most general case of the apparatus of FIG. 1f the location of the antinodes may or may not be the same as that of the transducers 6c. Further, the number of such nodes may be different than the number of transducers. Further, the nodes and antinodes may be moved around (to achieve a scanning effect) as by varying driving frequencies or phases of one or more transducers 6c.

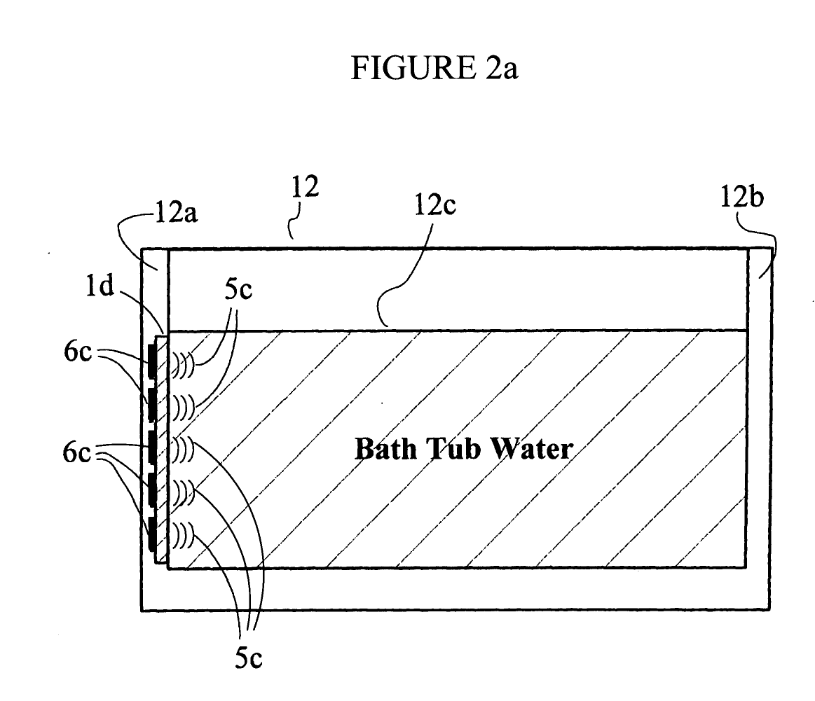

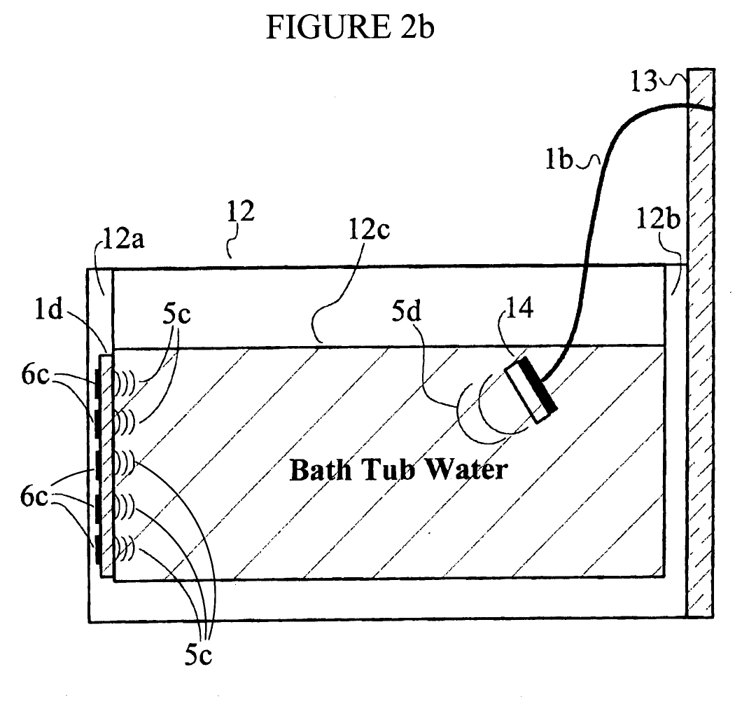

Moving now to FIG. 2a we see in a long sectional side-view a full bathtub, Jacuzzi® hot tub or other immersion container or tub 12 for a patient (patient not shown in tub). We note that tub 12 has a left end 12a and a right end 12b. The tub 12 is depicted as being mostly full of water 12c. On the left end 12a can be seen an inventive waveguide plate 1d having five transducers 6c back-mounted on it similar to those of FIG. 1f. We note that waveguide plate 1d in this example is acting as a portion of the tub 12 itself in that it helps contain water 12c. Alternatively it could operate through bulkhead 12a or from inside bulkhead 12a. In the FIG. 2a example shown the membrane 1d is also a water-containment feature such that transducers 6c are situated on the outer dry face of 1d. Thus transducers 6c are electrically isolated from the patient (patient not shown in tub). In practice, the transducers 6c would also preferably be covered by waterproof insulation and covers such that splashes or tub overflow still cannot cause an electrical shock hazard. Note that waveguide plate or membrane 1d is depicted as transmitting five antinodes of acoustical energy 5c into the tub water 12c. Waveguide plates 1d with associated excitation transducers of the type 6c could be arranged on any tub wall or even on the tub bottom. Further, we include in the scope the waveguide plates or membranes 1d having curvatures to achieve a design function such as to create at least a portion of the inner surface of a curved bathtub or to physically focus or direct one or more transducer emissions. In the case of curved plates or membranes 1d it may be preferred to edge-excite or end-excite them (as in prior figures for example) as it can more challenging to back-mount transducers to a curved nonflat plate.

So membrane/transducers 1d/6c of FIG. 2a may be operated simultaneously, in an interleaved manner, in a phased manner such as to achieve steering or as a subset of those available. Further, as stated earlier, our membrane 1d may be excited into one or more sequential, interleaved or simultaneous modes by one or more transducers 6c. Included in the scope is the membrane itself being made of transductive material—i.e., a material which itself is capable of producing acoustics. The plate or membrane may be rigid, semirigid, flexural or even elastically tensioned. Acousticians will be aware that any of these can be acoustically excited to emit energy into the water 12c. Typically, but not necessarily, the membrane 1d will be air-backed on the dry side as shown to maximize acoustic coupling into the water 12c. Acousticians will know that by air-backed we mean “air-equivalent” wherein air, a vacuum, or a foamlike material of very low impedance causes a beneficial backwards-going high acoustic reflection coefficient. So, in summary, the membrane or plate 1d may be curved or flat and may have one or more transducers or acoustic output ports coupled to it from one or more locations (5 locations shown). Typically the transducer(s) such as 6c will not completely cover the plate/membrane 1d backside such that the plate/membrane acts as a plate, shell or membrane whose own stiffness and density substantially determines the acoustic modes that can be delivered by firing one or more transducers 6c. Typically plate or membrane 6c will have a complex set of waves traveling laterally in the plate as well as being leaked-out of or emitted from the plate/membrane. Again, this is analogous to experimental “panel-speakers” comprising a plate with one or more transducers coupled to it and acting as an audio speaker. We include in the scope of the invention the use of the apparatus to also (or even instead) provide audio sounds or entertainment to the treatment subject. We note that one may advantageously carefully control the way the plate/membrane is attached at its edges as that coupling will affect the modes that can be excited therein. Varying the water level will also affect the mode states and monitoring and control of the water level is included in the scope. Finally, although not shown, we carefully include in the scope an acoustic power sensor in the tub or at some other convenient location which gives an idea of one or more parameters of the acoustic energy being experienced by the patient. This sensor may take any form and may also act as an interlock (to assure a maximum acoustic power is not exceeded or to assure the transducers are not operated when the tub 12 is empty or too-low in water level) or as part of a feedback loop to the apparatus control means.

Moving now to FIG. 2b we see basically the same apparatus as that of FIG. 2a except that we have added a handheld treatment applicator 14 containing its own waveguide 1b and shown emitting its own acoustical energy waves 5d. The applicator 14 is shown connected to yet another acoustical waveguide 1b which is mounted to (or passed through) a wall or bulkhead 13. Wall 13 may also be a part of the tub 12 construction (not shown). An acoustic energy source (not shown) could be situated behind the wall 13, isolated from the patient, yet coupling acoustical energy 5d into the conduit 1b and its applicator 14. Again, we mention that our inventive apparatus will frequently utilize two or even more waveguides of the invention. The apparatus of FIG. 2b likely has two and possible even three waveguides.
1. The first is shown waveguide/membrane plate 1d for immersion energy input similar to that of FIG. 2a.
2. The second is shown conduit or waveguide 1b feeding the treatment applicator 14, similar to conduit 1 of FIG. 1 for example.
3. The third is inside the applicator 14 itself, depicted schematically, and could comprise a small waveguide plate fed by the conduit 1b. This is similar to FIG. 1b wherein we have an acoustic energy waveguide feeding a vibrating acoustic window or could also be similar to FIG. 1d or 1e except that the transduction means in those figures is replaced in FIG. 2b applicator 14 by an acoustic energy output port (the output of conduit 1b of FIG. 2b).

It will be noted that in the preferred arrangement of FIG. 2b the patient is not exposed to any shock hazard even if the patient utilizes the applicator 14 underwater. This is because we have utilized acoustic waveguides (1d, 1b and the possible additional one in applicator 14) to electrically isolate the patient. As mentioned earlier, an applicator of the type 14 could also be used above water 12c as by providing the applicator with a wettable or gel-coated couplant layer or standoff. The acoustic treatment delivered by the different waveguide means described may be of the same spectral content or may be different, may be simultaneous, sequential or interleaved, and may be delivered automatically or with some amount of practitioner or user (treatment subject) direction or input.

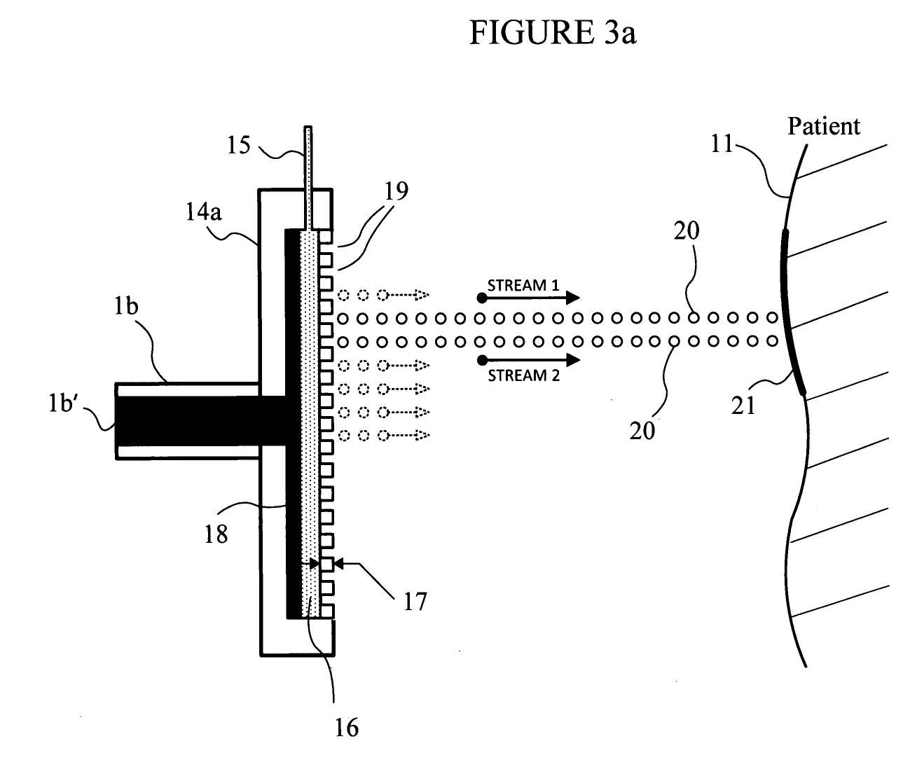

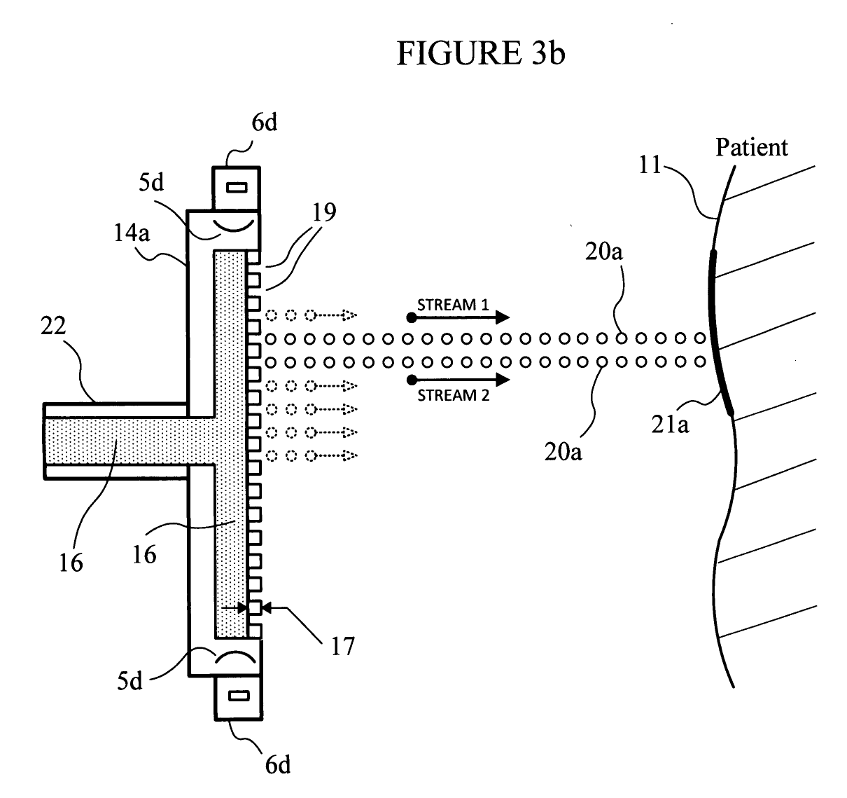

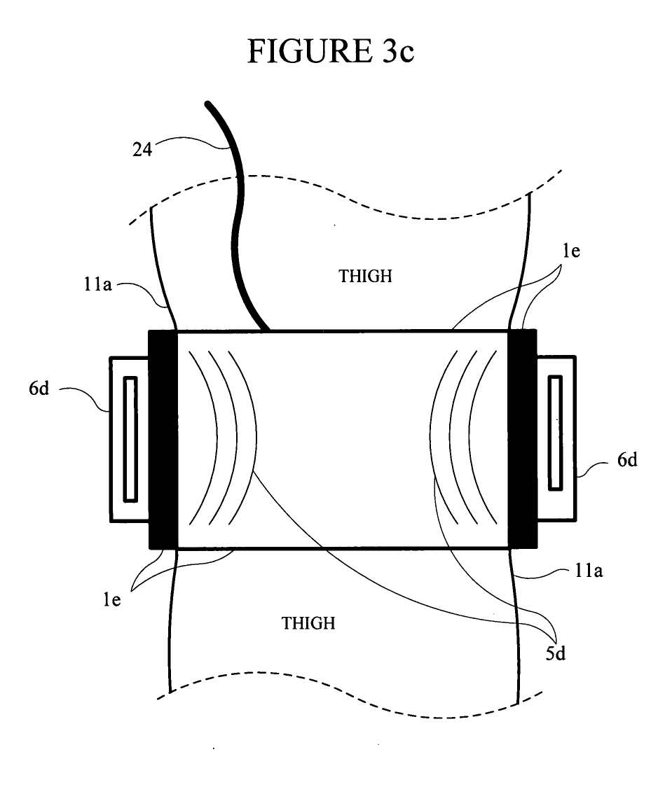

Moving now to FIGS. 3a and 3b we will describe a water-jetting or streaming variation of the inventive apparatus. Miwa, cited above, teaches the delivery of fat-treating ultrasound through a showerhead incorporating a transducer. In our FIG. 3a we see an improved version of a showerhead-type apparatus. A showerhead or handheld applicator 14a is shown. The applicator 14a is depicted as emitting essentially continuous streams of water 20 (only two shown) rightwards to impact upon patient 11 in an impact skin-region 21. The applicator is shown having a water aperture or orifice plate 17 from which streams such as 20 emanate from plural orifices or nozzles 19. A water-chamber 16 is pressurized or suitably fed by an inflow tube 15 such that water is forced out of orifices 19 of aperture place 17. Also shown is a familiar waveguide plate/membrane 18 inside the applicator 14a being excited by an incoming sheathed acoustic waveguide conduit 1b′/1b. The sheath portion is depicted as item 1b and the core portion as item 1b′. A nonsheathed guide is also in the scope. Thus, ultrasonic energy waves travel along conduit waveguide 1b′/1b to excite waveguide plate 18 which in turn directs its acoustical energy rightwards in a manner ultrasonically exciting waves which can pass out of the orifices 19 inside the more or less continuously emanating water streams 20. Thus we have in FIG. 3a up to three waveguides per our definition.
1. The first is conduit waveguide 1b′/1b delivering acoustical excitation energy into continuously streaming applicator 14a.
2. A second plate-type waveguide 18 which in turn passes that energy rightwards into the water-chamber 16 and then through the orifices 19.
3. Third stream-type waveguides 20 whereupon the acoustically excited streams impact skin 21 of patient 11 and deliver acoustical treatment or therapy.

Our waveguide conduit 1b′ is useful for electrically isolating applicator 14a. The waveguide plate 18 is useful to spread and distribute that energy across the entire face of the aperture or orifice plate 17. Per our previous teaching the waveguide plate or membrane 18 may be excited by a waveguide 1b/1b′ (shown) or by one or more coupled transducers in a manner allowing for one or more membrane modes to be excited.

As an example, waveguide plate 18 could be excited in a low or first drumhead mode or a simple piston-mode by the single centrally coupled incoming waveguide 1b/1b′ (shown). Alternatively, one or more waveguides or transducers could excite membrane 18 in one or more membrane modes depending on where the waveguides or transducers are mounted/coupled and how they are operated individually or together. Practitioners of ultrasound will note that it would be preferred not to have aperture plate 17 absorb a large amount of the incoming acoustical energy such that a reduced amount is able to progress along waveguide streams 20. Such practitioners will realize that one could take several measures to enhance energy-efficiency such as by combining aperture plate 17 and waveguide plate 18 into a single plate or by making a separate aperture plate 17 to be very stiff and/or have very loss losses. It is the employment of engineered waveguides of the type 1b′/1b and 18 that we consider novel in FIG. 3a as these allow for the delivery and distribution of acoustical energy over a region or volume of tissues 11/21 without having to have a large array of transducers upon or directly adjacent the patient.

We note a few further details in FIG. 3a. First, the acoustic energy passing between membrane 18 and aperture plate 17 may be resonant in the narrow cavity shown or may operate at a resonance of the applicator 14a or of a component thereof. In other words the water cavity 16 might be designed to be a resonant cavity or the aperture plate 17 might be designed to be a resonant layer or plate. In any even acoustic energy passes into streams of the type 20 and (at the speed of sound) propagates inside of said streams 20 to the patient 11 tissue impact region 21. Depending on the details of how membrane 18 is acoustically driven, one or more streams 20 will carry ultrasound either simultaneously or sequentially. That ultrasound will be delivered into the patient's tissues 21 for the useful treatment or therapy purpose(s). We note that one may have flowing streams (one or more) which have no propagating ultrasound for at least periods of time. Likewise the fluid flow of one or more streams 20 may actually be stopped or started selectively as may be the acoustical propagation along one or more streams 20. Finally (not shown), the streams 20 may impact tub water near the immersed or wetted patient and still deliver useful therapy through any intervening water path. Obviously one could deliver a variety of soaps, detergents, wetting-agents, cavitation-controlling agents or even topically-applied medicaments through one or more streams 20 (or via tub waters 12 of earlier figures).

Moving now to FIG. 3b we see an apparatus similar to that of FIG. 3a yet fundamentally different because in FIG. 3b the liquid waveguide streams 20a are comprised, at least in part, of spaced droplets which impact on a patient 11 tissue region 21a. Because droplets arrive at an arrival frequency they excite that frequency on and under the tissue 21a of patient 11 by simple kinetic energy loss. The arrival frequency is determined by the droplet spacing and the average droplet velocity. The acoustic intensity experienced by tissue 21a and thereunder is determined by the droplet kinetic energy which is a function of droplet velocity and mass. The acoustic power is determined, beyond the previous factors, by the arrival frequency of droplets having said kinetic energy. It will be noted that the apparatus of FIG. 3b also cools the tissue as does that of FIG. 3a assuming the impacting water droplets are cooler than the acoustically treated tissues such as 21a and at-depth. We include in the scope temperature control of emanating streams of FIG. 3a or 3b for purposes of cooling or heating the patient's tissues for any therapeutic or comfort reason.

We note in FIG. 3b that we again have an applicator head 14a with a frontside aperture or orifice plate 17 with orifices 19. In the case of FIG. 3b water 16 is fed into the applicator 14a through tube 22. We note that two transducers 6d are shown mounted on the edges of applicator head 14a and aperture plate 19. Those familiar with the acoustic arts and droplet-formation will be aware that it is widely known that an otherwise continuous stream of emanating liquid can be controllably broken into uniform droplets by acoustically exciting the exit-aperture or orifice(s) 19. In particular, a method frequently utilized in molten solderball-deposition research and in high-speed continuous inkjet printing is to transversely excite the orifice. In that manner we have transducers 6d passing acoustic waves along aperture plate 19 such that the orifices 19 are vibrated sideways (normal to the exiting stream 20a). This encourages the droplets to be formed at that frequency for each such excited and water-fed aperture 19. We stress to the reader that the apparatus of FIG. 3b, as shown, has just one waveguide type and it is the droplet stream(s) 20a itself. (Refer back to waveguide definition.) We believe that the delivery of such droplet streams at therapeutically useful acoustic frequencies is novel regardless of the exact arrangement of orifices, orifice pressurization, and orifice excitation. The reader will also be aware that small droplets traveling at high velocity will be somewhat slowed by ambient air—if any. The smaller the droplets and the higher the velocity the greater the slowing as is known by those designing high speed continuous inkjet printers. A secondary phenomenon is droplet fragmentation wherein the droplet is ripped apart by turbulence forces. Thus one generally would prefer to have short throw-distances to minimize slowing and fragmentation. Within the scope of the invention are preferred throw-distances ranging from approximately zero up to a meter or so.

It can be seen that a regularly spaced series of droplets (as shown) passing rightwards at velocity V meters/sec with a droplet packing density of N drops/meter will excite an acoustic wave at the point of impact having a frequency of VN droplets/second or VN hertz. So if one desired to excite a 150 kilohertz wave at the point of impact one could choose one of the following combinations if we assume, for simplicity, that droplets are spaced by gaps of one drop-diameter in size:
Velocity 1 meter/sec
Drops/meter 15,000
Effective Frequency 15 kilohertz
Velocity 10 meter/sec
Drops/meter 15, 000
Effective Frequency 150 kilohertz
Velocity 1 meter/sec
Drops/meter 150,000
Effective Frequency 150 kilohertz
Velocity 10 meter/sec
Drops/meter 150,000
Effective Frequency 1.5 megahertz

Note that for equal drop spacing and diameter the drop size scales proportionally to 1/(#drops/meter). The drop size does not necessarily have to scale with the spacing-this is just a simple example.

We also note that one may choose to deliver the droplets with the help of blown or forced air or gas. In that case one may choose to create the droplets, at least in part, using known aspiration nozzle techniques (optionally using our inventive membranes therein). Thus one could acoustically create droplets or utilize conventional nozzles to form the droplets. The invention is not tied to any particular way of droplet-creation however we prefer acoustical techniques since a high-quality stream can be created with excellent control. The embodiment of FIG. 3b thus centrally focuses upon the idea of treatment or therapy utilizing acoustics caused by impacting arriving streams of spaced droplets. Those familiar with droplet research in the arena of molten solderball delivery (the droplet therein is molten solder heading for a circuit board lead for example) will also be aware that droplets can also assume arrangements having something other than a fixed spacing-such as traveling packets of droplets which are widely spaced from each other but still arrive in rapid succession. The acoustician will realize that the type of ultrasonic energy excited in the patient 11 at and under tissue 21a for such a packetized stream would be akin to pulsed CW or pulsed continuous wave. The short continuous wave is formed by the packet droplets and the pulse delay between arriving packets is that related to the gap between arriving packets.

It will be appreciated that the droplet impact energy is the droplet kinetic energy or (½)MV² from basic physics and we can see from the earlier droplet spacing, velocity and size choices above that one can easily achieve a huge range of kinetic energy per droplet (by varying mass and velocity) and acoustic power (by further varying droplet lineal density).

We anticipate the possibility that the patient may desire to wear a protective layer such as a thin rubber or urethane coating to reduce the potential stinging effect of arriving droplets of very-high energy. The appropriate material, such as low-loss urethane, will not appreciably attenuate the acoustical energy. Such a coating could be in the form of a snug-fitting or elastic garment, perhaps disposable. One could also consider the use of a gel on the tissue to ameliorate impact sensation. By minimizing the throw-distance it is clear that one can deliver useful acoustical energy to the tissues with sufficiently high velocity and/or droplet size. Those familiar with droplet formation using ultrasound will also be aware that most techniques cannot easily produce droplets of diameter D spaced by gaps of dimension D. More typical is droplets of diameter D spaced by gaps of 2-5D and this is partly because of surface tension phenomenon and conservation of mass during stream-neckdown. Our FIG. 3b depicts droplets of diameter D spaced at approximately 1.5-2.0D which is easily doable using vibrating apertures (shown) or pulsatile pressure application (not shown in FIG. 3b) from an acoustic source upstream (behind) the aperture. Pulsatile pressure application could be provided as by our inventive membrane, a prior art transducer or any-other known pressure pulsation means.

Droplets may be delivered as a continuous stream with a fixed frequency of arrival and constant droplet/droplet spacing or may be delivered as a packet or packets of closely associated droplets which are spaced further apart from each other. A constant stream of equally spaced droplets would act like a continuous wave or CW acoustic excitation at tissue 21a of FIG. 3b. A group of spaced droplets packets would act like pulsed CW ultrasonic excitation. It should be clear that the embodiments of FIGS. 3a and 3b could replace conventional transducers which require some type of physically-contacting acoustic coupling during operation. It will also be apparent that the droplet apparatus of FIG. 3b could have multiple droplet streams 20a which are parallel or are not parallel and which are synchronized or not synchronized in some parameter. The operation of one or more droplet streams 20a might be continuous, discontinuous, interleaved or sequential, for example. The apparatus of FIGS. 3a and 3b may also be implemented such that a drug or medicament is delivered in the stream(s) itself or such that the stream water or other liquid is heated or cooled such that it heats or cools the patients tissues for bodily temperature control or for implementing a thermally-driven (hot or cold) lipolysis process included-herein by the cited references, for example. We note that in some cases the apparatus of FIG. 3b will have very fast droplets which can even cause a stinging sensation or irritation on the skin. We again specifically include in the scope of the invention the patient wearing a protective garment or coating such as a gel to avoid this. The garment could, for example, comprise a very this stretchable urethane or rubber film which is wetted or gelled underneath for good acoustic contact to the skin. Likewise, a skin-coating such as a cream could be used provided it is resistant to being washed off by the water during therapy. Included in the scope is any of our continuous or discontinuous streams delivering useful abrasion, descaling or cleaning action.

It will be recognized that for the apparatus of FIGS. 3a and 3b the treatment region of the patient is defined by areas or points addressed by impacting streams or droplets. The ultrasound produced at points-of-impact will emanate from that point into the tissue both depth-wise and laterally as pressure-waves and as shear-waves in known manners. Given a relatively low arrival frequency of less than 200 khz the tissue acoustic attenuation will be quite low and any target tissue lying between the impact points of individual streams will experience some of the ultrasound as it spreads out laterally. Thus it is not necessarily required that every square millimeter of the skin-surface be hit with a stream directly, only that the stream impact points are close enough that such bridging of ultrasound in the tissue can take place. We prefer an arrangement wherein a stream or droplet of diameter D is spaced from its neighbor by a gap of D to 10D with a 1D-3D gap being preferred. We note that if the patient moves relative to the stream (regardless of which apparatus-component or patient body actually moves or scans) then the tissue can be scanned by having a stream-pattern (or even single stream) passed over the tissue. We specifically include in the scope of the invention all manner of scanning the continuous 20 or droplet 20a streams over the tissue including applicator movement, patient movement and apparatus scanning mechanisms such as electronic beam-steering or mechanical translation or rotation scanners. Keep in mind that such scanning could be done by either or both of a fixedly mounted transducer or a handheld applicator.

Preferred frequencies of operation for the various apparatus of the invention are in the range of 1 hertz to 1.5 megahertz, more preferably in the range of 1 kilohertz to 600 kilohertz, and most preferably in the range of 25 kilohertz to 160 kilohertz. One may employ a fixed frequency, a variable frequency, multiple frequencies, a narrowband frequency range, a broadband frequency range, or a scanned frequency.

In the particular case of the droplet apparatus of FIG. 3b such droplets may be spherical, teardrop-shaped, ellipsoidal, spheroidal, rod-shaped or disc-shaped for example and this depends on the droplets interaction with the ambient through which it is propelled as well as how and how long(far) ago it was emitted. Those familiar with droplet science will realize that soon after emission a droplet takes a quasi-equilibrium shape as a function of turbulence forces and in-drop oscillations. Inventive droplets will typically be between 1 micron and 5000 microns in diameter, more preferably between 5 microns and 2000 microns diameter, even more preferably between 10 microns and 500 microns diameter, and most preferably between 20 microns and 200 microns in diameter. As far as droplet spacing is concerned, streams 20a can be formed of droplets of diameter D with inter-droplet spacings or gaps of 1D, 2D, 3-5D, 6-15D an even 16-100D and more. Preferred droplet spacing is in the range of 1D-15D. Those familiar with droplet formation will be aware that droplet spacings of 3D-10D are quite common in experimental microdroplet emitter experimentation and are easily achieved using acoustic disruption techniques as depicted in FIG. 3b.

For the droplet apparatus of FIG. 3b the ultrasonic excitation frequency experienced by the patients tissue 21a (and beneath that surface depending on any attenuation and mode-conversion) will be, at least in part, determined by a combination of droplet velocity V and droplet spacing X as by a product of V times 1/X, or V/X. Further, it will be recognized that the acoustic intensity of the droplet apparatus of FIG. 3b will be determined by, at least in part, the droplet mass M (a kinetic energy factor) and droplet velocity V squared (a kinetic energy factor). The average power will also take into account droplets impacting over time having in-flight inter-droplet spacing X. Thus the average acoustic power from a droplet stream will scale with (VM)/X.

The patient or treatment subject for any of the apparatus or methods may be entertained before, during or after an actual therapy or treatment delivery. Given that attractive feature we expressly include in the scope of the invention the apparatus incorporating, integrating or being used in cooperation with an entertainment device such as an audio or audio/video device, internet-surfing device or radio for example. W have already mentioned prior to this our inventive membrane(s) as being capable of also acting as audio speakers—even underwater.

Moving now to the final figure, FIG. 3c, we therein see a preferred embodiment of the invention which can be strapped on a patient's or treatment-subject's thigh for possible home-use. Seen therein is a patient's thigh portion 11a around which an inventive waveguide based treatment apparatus 23 is fastened or urged thereupon to be in good acoustic contact with said thigh portion 11a. An acoustically-excitable plate or membrane-type waveguide 1e is seen wrapped-around some or all of juxtaposed thigh tissue 11a and it has shown mounted upon it two excitation transducers 6d. The transducers create acoustic wave-energy 5d which enters the tissues in one of several possible manners. It will be noted that the apparatus 23 has a power cord 24 attached thereto which runs to a power source, likely an electrical power source.

Acoustic waves 5d, can arise in several ways such as a) by passing from transducer 6d through the plate 1e and directly inwards, b) by passing from transducer 6d into plate 1e and then emanating from the plate, at least in-part, at a different location on the plate 1e, c) by acoustic leakage of in-plate waves that become laterally escaping waves. The reader will realize that transducer(s) 6d might be a pressure-mode or shear mode device for example. Preferably the transducer(s) 6d excite waves which travel in the plate waveguide 1e and are passed out or leaked out at several antinodes as per prior embodiments. Like the aforementioned Omnisonics products the waves within the plate may be standing waves having nodes and antinodes for example. Because of the skin-coupling to the thigh tissue 11a such in-waveguide waves will leak into the tissue as one or both of pressure waves and/or shear waves. Thus the waveguide plate in this example acts to distribute and homogenize the treatment around the circumference of the thigh 11a. One may choose to vary acoustic frequency for example such that a varying selection of antinodes are created and/or moved along the waveguide. The transducer(s) 6d might also be adjustable for position of coupling to the waveguide 1e so long as the needed good acoustic coupling between transducer 6d and waveguide 1e can be reestablished-such as by a gel contact between them. One may also permanently mount one or more transducers 6d to a waveguide 1e. Again we emphasize that we preferably are exciting the membrane or plate 1e into one or more of its vibrational modes using one or more transducers 6d such that most or all of the membrane 1e emits acoustical energy into the thigh 11a. This is very different than simply applying the bare transducers 6d directly to the thigh 11a (not shown) because our inventive membrane or plate 1e allows for many more modes of vibration and allows for distribution of acoustical energy away-from and between transducers of the type 6d. Thus our approach allows us to treat large areas with smaller transducers or fewer transducers and lighter weight. Membrane or plate 1e of FIG. 3 could, for example, be a flexible sheet of low-loss polymer such as a polycarbonate or a low-loss flexible metal such as superelastic Nitinol™ metal alloy. The membrane may have one or more layers and features designed therein to enhance (or avoid) particular modal patterns.

In FIG. 3c we have depicted the therapy apparatus 23 as having its waveguide surface, shell, plate or membrane 1e mounted directly to the thigh tissue 11a. Based on our previous apparatus and discussion the reader will realize that the apparatus 23 might be coupled to the tissue of 11a with a thin-coating of ultrasound-transmissive gel or cream for example. Likewise, the apparatus might incorporate a water-filled or gel filled or other acoustically transparent spacer or standoff (none shown under membrane 1e) between waveguide 1e and thigh tissue 11a. In terms of shape-adaptability and fit we note that in the case of an acoustic standoff or underside spaced/coupler being used it would be a simple matter to have that standoff comprise a flexible liquid or gel-filled bag or container which conforms to the shaped thigh for example. In the case of no acoustic standoff (as shown in FIG. 3c) one could also or alternatively make the waveguide 1e flexible such as by it being formed of, for example, titanium-based sheet or titanium-based wire braid or fabric as opposed to the above-mentioned polymer of Nitinol™. It also is not necessarily required that the waveguide 1e completely wrap around the thigh and close on itself such that it encompasses 360 degrees of the thigh 11a. For example, the apparatus may wrap 270 degrees around the fattest part of the thigh and have straps or clips that consume the other 90 degrees. The waveguide 1e may also have a spring-nature to it such that it can be forced open and allowed to close upon the thigh. We note that Nitinol™ titanium-nickel alloys mentioned above would serve this purpose nicely particularly as they are available both in superelastic and malleable form to enable an elastic or malleable apparatus 23.

The reader will also recognize that the apparatus 23 might include heating or cooling means for maintaining a body temperature or for driving a supportive, co-delivered or cooperating thermally-based therapy process. One convenient way of heating would be to heat a metallic waveguide 1e itself or heat a filler liquid or gel in a membrane-underlying acoustic standoff or spacer (not shown). Cooling could be done as by passing a coolant in contact with the waveguide 1e or cooling the liquid or gel contents of an acoustic standoff or spacer situated between waveguide 1e and thigh 11a. As for previous inventive embodiments, one may also drive a drug or medicament into the patient as by an intervening skin-patch or by putting the drug in a standoff liquid or gel or an underlying skin-wetting gel coating and allowing it to leak or perfuse inwards or be driven thermally and/or acoustically inwards into tissue.

The present inventors anticipate numerous permutations of the apparatus of FIG. 3c. For example, such a snap-on or conforming apparatus 23 could be placed on the thigh, on the buttocks, or on the belly. The apparatus does not necessarily have to be snapped-on, strapped-on or self-clamping. For example an apparatus for the buttocks could be sat upon. The apparatus might be worn during work, sleeping, play, jogging, running, exercising or being otherwise entertained or occupied as by reading. The required power-source, in the case of an electrical power source, could be provide by a wall-outlet, a battery or fuel-cell pack, a backpack power-source or a waist-band power source. In one special case one could have the patient exercise by running a generator which in turn powers the ultrasonic therapy device. This both forces and incentivizes the patient or subject to exercise and burn liberated (as well as unliberated fat constituents)-something that is vitally important if noninvasive fat reduction is to be accomplished.

An apparatus of FIG. 3c could be provided such that it can address a range of bodily sizes and shapes such as by bending or adjusting straps or clamps or by using different size or thickness standoffs or different or differently liquid-inflated standoffs. Alternatively, or additionally, the provision of various-sized apparatus 23 could be accomplished as by providing a selection of waveguides 1e with one or more common and preferably detachable driving exciters or transducers 6d. Thus we anticipate the possibility of a kit containing one or more of different apparatus, different transducers, different waveguides, or different couplers

Another potential means of attaching, or at least acoustically coupling the apparatus to the treatment subject, is by having suction-means pull the waveguide 1e (and/or coupling means) onto the tissue 11a or by having an inflatable member or inflatable-standoff push the waveguide 1e onto the tissue 11a or push itself into acoustic contact with an overlying waveguide 1e and underlying tissue 11a. Another approach is to have a garment which stretches elastically and when it is worn over the apparatus it provides the inward juxtaposition forces.

The present inventors prefer the apparatus of FIG. 3c to have at least a thin conformable acoustic standoff layer (not shown) between the tissue 11a and the one or more waveguides 1e or transducers (or acoustic-input conduits) 6d. This provides size-conformance as well as excellent thermal heat-exchange and allows for the waveguide to be somewhat less flexible and more inexpensive. Recall that even with such a standoff it is preferable that the tissue being treated be wetted as by a liquid or gel (or sweat) such that the standoff itself makes intimate acoustic contact. Thus a combined waveguide 1e and conformable standoff could be provided as by a liquid or gel-filled bladder. We include in the scope of the invention the case wherein, for any of the embodiments, a waveguide is a liquid or gel-filled container (like our previously mentioned standoff) and it has acoustics coupled into it.

The present inventors have described some preferred waveguide materials such as titanium shells, strips, wire-braids, Nitinol™ sheets or braids and polycarbonate (polymeric) sheets, strips or braids. We have also referred to a liquid-filled or gel-filled waveguide. A liquid filled-waveguide could, for example, comprise a water-filled annulus or torus which fits snugly or is inflated around the target limb. The transducer or transduction means such as 6d could be coupled into such a liquid or gel type waveguide in any desired manner including by placing one or more transducers inside the liquid or gel itself.

For any waveguide of the invention including that of FIG. 3c, the inventors note that acoustical energy may be driven around a closed version (completely wrapping and self-joining) of such a waveguide such as a contiguous water-filled torus or a continuous Nitinol™, aluminum or titanium film such that it acts to store significant acoustic energy available for leakage or other redirection into the adjacent tissue. In that manner circularly resonant modes become available and losses due to end-reflections might be reduced. For waveguides which do not completely enwrap the thigh one can utilize the finite ends thereof for purposes of creating useful reflections, antinodes or nodes at the ends. In the case of a membrane 1e overlying a gel (or liquid) filled standoff then the acoustician will appreciate that this is a dual-layer system having a set of modes which depend both on the membrane/plate and on the thickness/shape/dimensions of the underlying standoff layer. In all cases we have at least a single-layer membrane or plate driven into one or more membrane modes by one or more transduction means.

Also included in the scope of the invention is the custom fabrication or fitting of an apparatus or any part thereof-such as a custom fitted waveguide or acoustic standoff. One could utilize laser-scanning devices on the patient's body to determine the needed shape. This is similar to custom ski-boot fitting.

We have cited references pertaining to drugs and diets useful for obesity-reduction and/or weight loss. It is our intention that our inventive apparatus and method may utilize one or more such drugs, medicaments or diets in cooperative association with our apparatus and method and as part of said inventive apparatus and method. Such drugs, medicaments or diets may be administered or undergone before, during or after at least one such therapy or treatment session. Our references included genetic-based and stem-cell based drugs and therapies which we consider drugs herein. Note that our apparatus is employable for maladies other than fat-reduction and obesity and we explicitly include in the scope drugs, medicaments and diets for those. An example would be ultrasonic therapy for burned skin which also utilizes a surface ointment or drug—whether or not it also undergoes acoustically-assisted skin-penetration.

For application as an energy-conduit or waveguide such as conduit 1a we described a sheathed or unsheathed wire or rod, perhaps made of titanium as is known to be suitable for Omnisonic's type referenced devices. The conduit may have a single wire within or several wires and multiple such wires may be spaced as by additional low-impedance acoustically-reflective material. We also include in the scope the use of a liquid-filled acoustic waveguide. Such a waveguide would have an acoustically reflective sheath such as a metal of high acoustic impedance or an air-filled material of low acoustic impedance. As far as we know such a liquid filled waveguide is also novel—particularly for an acoustic therapy apparatus of the invention.

We include in the scope of the invention the use of imaging machines to view the patient's body or targeted tissues before, during, or after delivery of at least one such therapy or treatment. Radiology practitioners will be aware of useful tools for this purpose such as X-ray machines, fluoroscopy machines, MRI machines, CATSCAN machines, PET machines, SPECT machines and ultrasound imaging machines for example. Coming in the next few years also are terahertz imaging machines and optical-coherence-tomography or OCT imaging apparatus. One may, particularly for the delivery of the higher acoustical power mechanisms such as cavitation or necrosing, desire to do real-time imaging. Thus we include in the scope of the invention the placement of an immersion container of the invention within such an imaging tool (or vice versa) with precautions being taken to assure that one does not, for example, put magnetic materials inside an MRI machine. The reader should again note that some treatments or therapies providable by the inventive apparatus may involve tissue-heating, tissue-cavitating or acoustic-streaming inducing acoustic irradiation. The streaming mechanisms are frequently used for drug delivery.

We further include in the scope of the invention the use of exercise apparatus before, during, or after delivery of at least one therapy or treatment of the invention. In fact one may incorporate an exercise apparatus within (or within reach) of an immersion container or applicator of the invention.

We have also described at least a couple of invasive and semiinvasive surgeries such as stomach-stapling and vagal-nerve ablation for difficult obesity cases and we include the use of such surgeries in combination with our described treatment or therapy as well.

Either or both of an immersion apparatus or an immersion or non-immersion applicator may be utilized in an actual bathtub, shower or Jacuzzi® for example. In such applications we include in the scope of the invention any ultrasonic-cleaning benefit that may be derived from having the therapeutic ultrasound present.

The present inventors are also interested in the application of the inventive apparatus and method in home-use treatment or therapy devices which do not necessarily involve a bathtub or Jacuzzi®. We broadly define immersion to mean that a body of liquid, gel or other flowable or formable acoustic couplant or coolant at least conforms-to or surrounds a body member to be treated. So, as an example, one could have a thigh-worn apparatus which includes a waveguide apparatus of the invention. Such a snap-on apparatus could employ a body-shaped or body-shapeable waveguide or could employ a conforming water or gel standoff with a nonconforming transducer or transducer/waveguide mounted thereupon. In all cases of our inventive apparatus and method we have at least one waveguide/membrane member. In the case of the thigh-mounted slimming apparatus one could have a formable metallic sheet-like waveguide of titanium excited by at least one back-mounted or edge-mounted transducer. The formable or wrappable waveguide may or may not be spaced from the thigh tissue with a water or gel standoff spacer to afford further comfort, size-adaptability or require less formability of the titanium sheet waveguide. One might sell or offer a variety of sizes of any one or more of excitation transducers, waveguide-sheets or plates, or water/gel standoffs or couplers. Any one or more of these may also be arranged to be disposable as could be a gel for coupling the apparatus to the tissue.

A compact thigh-mounted, abdomen or belly-mounted conforming waveguide apparatus could even allow for the patient or treatment subject to walk, run, swim, exercise or sleep during treatment. One might place the power supply for the transduction means in a backpack or waste-belt for example. Obviously, such apparatus could also be used on a bedridden person or a person simply lying in bed or on a couch. The person could be entertaining themselves while undergoing therapy as by watching television, reading, or listening or watching music and/or audio.

Included in the scope of the invention is the apparatus having some software to control it and/or enable its use on a particular person. For example, given a network or other wired-connection, one could have a remote computer or person authorize or review potential treatment subjects for safety, for suitability or for therapy progress. The user may operate the apparatus himself/herself perhaps with a remote authorization based on a recommended or authorized therapy, based on a credit statement or based on an online payment.

One may also choose to integrate the use of vital-sign(s) monitoring devices such as blood-pressure or heart-rate sensors and instruments to assure that the patient is in good condition given where he/she is in his/her therapy. It is known from our cited references that FFAs (free fatty acids) are known to increase upon successful instigation of adipocyte-destruction or degradation or upon the encouragement of natural lipolysis mechanisms (Miwa references). We fully expect the noninvasive or minimally invasive ability to measure FFAs in the future and recommend such a sensor be used with the apparatus at least in cases wherein potentially dangerous quantities of FFAs or other adipocyte-destruction byproducts (adipocyte connective tissue, adipocyte-surrounding blood vessels) are to be absorbed by the body. It will be obvious that the inventive apparatus could utilize the data from one or more such sensors to modulate, enable, disable, interrupt or control a therapy-preferably in a closed-loop algorithm which does not require the patient to make any such safety or dose (if applicable) decisions.

The drugs and medicaments which we have referenced may alternatively be delivered by placing them in an inventive immersion liquid or within an acoustically excited (or not) liquid emanating from an inventive applicator. They may also be placed in a skin-patch or in a liquid or gel standoff from which they are driven into tissue by the therapy ultrasound or pass into tissue by thermal or simple concentration-gradient reasons. To do so we can provide a permeable or hole-pocketed gel or liquid container—or even an underlying skin-patch. Such a skin-patch may also function as our acoustic spacer or standoff and may come with medicaments already provided therein or thereon. We include in the scope the idea of operating such a transducer(s) in two separate modes simultaneously, sequentially or in an interleaved manner, one mode to deliver acoustic tissue-therapy and another mode to drive the drug inwards and/or activate the drug in-vivo or in-vitro. Thus the therapy ultrasound apparatus may also serve to activate or enable the work of the drug once it reaches its target depth. One mode may also suffice for both purposes.

Several techniques exist to utilize ultrasound, heat, electrical potential or other energies or fields to drive medicaments or drugs through tissue-such as through the skin from the skin-surface. U.S. Pat. No. 6,527,716 to Eppstein, US 20020156414A1 to Redding and WO0002620 to Zhang provide a good set of references on this technology. We note that since we have ultrasound and heating/cooling capability available in many of our embodiments it would be a simple matter to utilize any of those energies, fields or gradients to urge a drug or medicament into the tissue-perhaps directly under our applicator head for example. Again, the drug might be provided as part of an acoustic coupler or standoff such that it leaks or leaches into the skin through a membrane making up the standoff. It might also be provided as a skin-patch or treated skin-area under our applicator or may alternatively be applied away from our applicator area as by an independent means taught in the above three prior art patents. Another possibility would be to have an immersed sonicated patient be immersed and exposed in water which has a drug or medicament mixed in with it such that by the combination of exposed immersion and immersed sonication the patient has the drug driven into or diffused into his/her skin and body by at least one of those exposures and/or sonications. We have also previously mentioned that even a handheld applicator or a strap-on treatment apparatus could deliver a drug in a similar manner. Note that it is the use of such drug-delivery techniques in combination with our apparatus that we claim and not just the subcase wherein, for example, an electroporation device, a drug-filled standoff device or a drug skin-patch is integrated directly in our own applicator.

We have cited several prior art mechanisms for the specific addressing of adipocytes. We note that the reported frequencies and acoustic power-levels naturally vary depending on whether one wishes to heat or cavitate or alternatively, stimulate lipolysis at very low acoustic power levels. The same applies to whether or not the ultrasound is pulsed or continuous wave for example. It will be seen that most of the adipocytes-depleting or destroying art cited describes frequencies of operation in the range of a few kilohertz to 200 kilohertz with one or two as high as 1.0-1.5 megahertz. The Miwa reference also teaches staying below known mechanical indexes indicative of cavitation and thermal indexes indicative of tissue necrosis if low power ultrasound is being used merely to stimulate natural lipolysis processes as opposed to cavitationally or thermally destroying adipocytes-related structures. Thus, depending on which of the referenced mechanisms one chooses to implement with our inventive apparatus, one may choose one or more of the following: a) an energy or energy-density above a cavitation or cavitation-index threshold, b) an energy or energy-density above a thermal-index or thermal-damage threshold, c) an energy or energy-density known to damage or disrupt any portion of an adipocyte's or surrounding connecting tissue or blood vessels, d) an energy or energy-density known to disrupt an adipocyte's membrane or an adipocyte's phospholipids layer or to cause the release or activation of a lipolysis related hormone or enzyme, e) an energy or energy-density at or below a regulatory limit set for diagnostic ultrasound, f) an energy or energy-density known to indirectly disrupt adipocytes via causing damage to their surrounding connective tissues and/or microscopic blood vessels, g) an energy or energy-density which requires cooling of a tissue surface in order to prevent the ultrasound energy from thermally damaging the tissue surface, h) an energy or energy-density which is utilized to support the movement of a supporting drug through the patients tissue or body or i) an energy or energy-density that promotes therapeutic tissue heating to accelerate lipolysis, metabolism or the action of a beneficial drug.

Any of the inventive apparatus may beneficially be operated using or having one of the following acting in supportive cooperation: a) a treatment timer, b) a timer which interrupts treatment upon completion, c) a treatment software or firmware program or algorithm, d) a treatment selected from an available selection of treatment programs or algorithms, e) an emergency-off button or switch, f) a safety interlock which recognizes a treatment subject's distress, g) a computer or internet network-connection over which at least some data, information or status passes or can pass, h) a disposable which is required to activate the apparatus—such as for filling a gel into a gel standoff, i) a password or patient-identifying piece of information being required for use, j) a treatment which is one of an authorized or purchased set of multiple treatments, k) a doctor's prescription for a cooperatively used drug or medicament or for a specific apparatus therapy, l) an anti-obesity surgery, m) an exercise program whose exercise activity takes place at any location and is not necessarily collocated (or fully integrated) and used while being treated by the apparatus, or an exercising means which may or may not power the treatment apparatus.

Any apparatus of the invention, particularly if it utilizes a liquid or flowable acoustic or thermal medium for any one or more of the taught purposes, may have such a liquid one or more of filtered, used once and disposed of, diluted, mixed, sanitized, treated for bacteria or fungi, recirculated or provided as a disposable. In any of these cases the liquid may be doped with a drug or medicament and said drug or medicament delivery means may be used together-with or cointegrated with the apparatus as part of the overall apparatus and method. A preferred disposable of the invention is a liquid or gel-filled standoff which is predoped with a drug or medicament to a needed concentration which aids the therapy process as the drug soaks into or is driven into the tissue through or across the standoff with or without the help of any heat or ultrasound presented by the apparatus.

As a final means of providing body-shape adaptability we note that one might utilize an acoustically transmissive material which is thermoformable by the heat of the body or by apparatus-generated heat. Such a material could, for example, be a thermoformable open-celled foam which becomes saturated with water for the required low attenuation

We include in the scope the treatment subject paying for his treatment or apparatus in any one or more of the following ways: a) a home-use unit is purchased or rented, b) a clinical visit is paid for, c) an internet-enabled payment system is used, and/or d) a prepaid credit-bearing card or memory-including dongle is used.

The use of cooling has been cited by the above prior art for purposes of crystallizing and killing the fat-contents of adipocytes. It is explained in reference US23220674A1 to Anderson, that the act of thermal crystallization itself is what destroys the fat molecules in the adipocytes. We herein extend the possible mechanisms of cooling approaches as follows. We believe that cooling, even before crystallization, hardens and stiffens the fat-contents as well as the adipocytes cell membranes. Given a vibrational excitation it follows that it would be easier to mechanically disrupt a stiffer less-deformable cell with such excitations. We therefore anticipate that cooling-induced stiffening makes cells more damage-prone to mechanical excitations. It should follow that mechanical agitation caused by phenomenon such as local cavitation or local acoustic compressive- or shear-waves would stand a better chance of causing physical damage to such stiffened entities. Thus we include in the scope of the invention the new damage mechanism of “damaging stiffened biological structures”. The mechanism should work regardless of whether such cooling-stiffening is cell-selective. We can assume from Anderson that at least the fat molecules will be selectively stiffened before they actually crystallize. A potential advantage of this technique is that one does not need to proceed down to crystallization temperatures but only down to a stiffening temperature. Included in the scope of the invention is the taking advantage of any existing stiffening selectivity among different biological entities as well as utilizing targeted ultrasound or vibrations to obtain a desired spatial selectivity. It will be recognized by the reader that this general mechanism might also be utilized to degrade or destroy diseased biological matter of any type.

We also note that it has recently been reported by the Washington University School of Medicine that it removed about 20 pounds of fat from each of 15 obese women and then tracked their heart-disease risk and insulin resistance over time. Contrary to the expectations of many, there was no improvement—only their appearance improved. What this result tends to say is that the fat which most hurts one's health is the deep-seated or visceral fat not usually addressed by superficial liposuction techniques. Given this finding we believe that our invention herein is capable of attacking deep-seated visceral fat with the same choice of mechanisms already listed. Liposuction techniques cannot address this deep fat.

We claim:

1. A wearable treatment or therapy apparatus for provision of an acoustically enabled or acoustically enhanced treatment or therapy to a patient or treatment subject comprising:
a) at least one acoustic transduction means or acoustic source;
b) a shell, plate, membrane or member serving at least as a lateral acoustic waveguide; and
c) a means to acoustically couple the apparatus to a patient treatment region,
wherein
1) at least one transduction means or acoustic source injects acoustical energy into said waveguide from upon or within said waveguide, the waveguide propagating at least some wave-guided acoustical energy laterally along at least one of its extended lateral dimensions;
2) said waveguide, in turn, passing at least some of said injected lateral energy into or toward said patient along or through the acoustic coupling means;
3) said waveguide thereby laterally distributing, redistributing, redirecting, storing, or causing acoustic mode modification of said at least some of said laterally injected energy before its passage toward or into the coupling means and patient; and
4) said waveguide adapted to conform to an as-coupled body shape and allowing for the delivery of acoustical energy into the patient or subject from substantially all of its surface including that surface area between the transducers thereby allowing for a few transducers to uniformly treat a substantially larger area than their own summed total area.

2. The apparatus of claim 1 wherein the treatment or therapy is for a purpose related to any one or more of: a) obesity treatment or surgery, b) fat reduction treatment or surgery, c) cosmetic fat reduction treatment or surgery, d) cosmetic reshaping of any type, e) invasive fat reduction of any type, f) noninvasive fat reduction of any type, g) modification or destruction of one or more adipocytes or their connecting structures, h) encouragement of a lipolyis process, i) delivery or activation of a drug or medicament supportive of a fat-related or obesity-related treatment or therapy, j) visceral fat reduction, k) shallow surface-related fat reduction, l) cellulite reduction or modification, m) a fat-reduction process requiring the subjects body to process and excrete a released or destroyed fat constituent, n) a fat-reduction process involving invasive fat removal, or o) home-use body shaping.

3. The apparatus of claim 1 wherein at least one of:
at least one said transduction means is a piezoelectric, piezocomposite, electrostrictive, magnetostrictive, electrostatic, ferroelectric, thermoacoustic, photoacoustic or electromagnetic transducer or is a laminate or active portion of a piezocomposite or piezolaminate; and
at least one said energy source is the output end or an output port of an acoustic conduit or waveguide.

4. The apparatus of claim 1 wherein at least one of:
a) the waveguide has integral to its structure at least one transduction means or acoustic source;
b) at least two different transduction means or energy sources are operated differently in time or in a driving-parameter in order to create a desirable acoustic vibration pattern in the waveguide;
c) at least one waveguide, transduction means or energy source includes a piezoelectric, piezoceramic, piezocomposite or piezolaminate, electrostrictive or magnetostrictive material;
d) a liquid or gel filled acoustic coupler is utilized;
e) the patient or practitioner can adjust or exchange apparatus components in a manner offering improved fit or formability;
f) at least a portion of the apparatus is disposable or of limited use;
g) the apparatus is strapped, clasped, fastened, tied, adhered, suctioned, buckled or otherwise held in intimate juxtaposition to the patient;
h) acoustical wave patterns having at least one of antinodes, nodes or traveling waveforms are created in the waveguide; and
i) the waveguide serves to spread treatment energy from one or more means or sources across a region of the patient tissue including to locations between or away from said means or source locations.

5. The apparatus of claim 1 wherein at least one of an apparatus portion or a patient tissue portion is controlled or monitored with respect to temperature.

6. The apparatus of claim 1 wherein the apparatus is utilized in cooperation with a drug, medicament or diet.

7. The apparatus of claim 1 wherein the apparatus is wrapped around a patient's anatomy to form a closed or joinable shaped apparatus, said wrapped apparatus either utilizing a fastener to be retained thereon or being self-retaining, at least the waveguide portion of the wrapped apparatus substantially conforming to the anatomy.

8. The apparatus of claim 7 wherein the specific wrapped anatomy includes one of a) a leg portion, b) an arm portion, c) a buttocks portion or d) an abdominal portion.

9. The apparatus of claim 7 wherein the apparatus is strapped or mounted in a conforming manner to the anatomy and a liquid, gel or flowable couplant is employed in any manner to acoustically couple the apparatus to said anatomy.

10. The apparatus of claim 7 wherein the apparatus contains multiple transducers and any one or more of:
a) multiple transducers are operated in synchrony or with controlled phase relationships;
b) one or more transducers inject acoustical energy, at least laterally, into the acoustic waveguide;
c) the waveguide redirects or passes at least some laterally propagating acoustic energy into the patient or subject; and
d) substantially all acoustic energy delivered into the patient or subject first passes through or along a waveguide lateral or thickness direction.

11. The apparatus of claim 7 wherein the waveguide is any combination of: a) substantially fabricated from titanium, an alloy of titanium, or a nickel-titanium alloy, b) substantially fabricated from a super elastic metal, or c) substantially fabricated from a material having low acoustic losses.

12. The apparatus of claim 7 wherein the wrapped apparatus allows for patient or subject ambulation.

13. The apparatus of claim 7 wherein the wrapped apparatus is powered by at least one of:
a) an ambulatory power supply;
b) a power supply connectable to the worn apparatus; or
c) one or more rechargeable batteries or fuel cells.

14. An apparatus for delivering an acoustic or acoustically-aided therapy or treatment to a patient or treatment-subject comprising:
an emitter of moving liquid droplets or flowable-material droplets;
a means for directing said moving droplets upon a patient treatment region;
the droplets traveling with an average droplet diameter, average velocity and average spacing at least at a moment in time;
the repetitive impact of said droplets upon said patient providing an acoustic excitation related to the droplet arrival frequency and an acoustic power related to the arrival velocity and arrival mass; and
said excitation passing into or along a patient tissue portion needing said therapy or treatment.

15. The apparatus of claim 14 wherein the treatment or therapy is for a purpose related to any one or more of: a) obesity treatment or surgery, b) fat reduction treatment or surgery, c) cosmetic fat reduction treatment or surgery, d) cosmetic reshaping of any type, e) invasive fat reduction of any type, f) noninvasive fat reduction of any type, g) modification or destruction of one or more adipocytes or their connecting structures, h) encouragement of a lipolyis process, i) delivery or activation of a drug or medicament supportive of a fat-related or obesity-related treatment or therapy, j) visceral fat reduction, k) shallow surface-related fat reduction, l) cellulite reduction or modification, m) a fat-reduction process requiring the subjects body to process and excrete a released or destroyed fat constituent, n) a fat-reduction process involving invasive fat removal, or o) home-use body shaping.

16. The apparatus of claim 14 wherein at least one of:
a) droplets are defined with the aid of an acoustic energy or transducer;
b) droplets are defined, at least in part, using a spray or atomization means;
c) droplets are formed, at least in part, using pressure pulses;
d) multiple droplets travel along a substantially same path to the patient;
e) droplets travel individually or as packets of droplets;
f) the apparatus is used in conjunction with a drug, medicament, diet or with a tissue temperature-controlling means;
g) the apparatus is for one or both of home-use or clinical use; and
h) the treatment subject wears or has an acoustically-transparent protective overlayer to shield some of the droplet irritation.

17. An apparatus for delivering an acoustic or acoustically-aided therapy or treatment to a patient or treatment-subject comprising:
an emitter of at least one moving liquid stream;
a means for directing said moving stream or streams upon a patient treatment region;
a means to inject acoustical energy into said stream or streams at the emitter end such that it propagates along said stream or streams toward the patient;
said injection means including or utilizing an acoustic waveguide in any manner;
at least some treatment acoustical energy contained in said moving stream or streams arriving upon or into said patient as a result of said stream or streams arriving; and
streams not necessarily remaining fluidically attached to said stream emitting means the entire time the stream and its contained acoustic energy impact upon the patient.

18. The apparatus of claim 17 wherein the treatment or therapy is for a purpose related to any one or more of: a) obesity treatment or surgery, b) fat reduction treatment or surgery, c) cosmetic fat reduction treatment or surgery, d) cosmetic reshaping of any type, e) invasive fat reduction of any type, f) noninvasive fat reduction of any type, g) modification or destruction of one or more adipocytes or their connecting structures, h) encouragement of a lipolyis process, i) delivery or activation of a drug or medicament supportive of a fat-related or obesity-related treatment or therapy, j) visceral fat reduction, k) shallow surface-related fat reduction, l) cellulite reduction or modification, m) a fat-reduction process requiring the subjects body to process and excrete a released or destroyed fat constituent, n) a fat-reduction process involving invasive fat removal, or o) home-use body shaping.

19. The apparatus of claim 17 wherein one or more emitters emit two or more streams and the waveguide is capable of providing acoustical energy to at least two or more streams any of sequentially, simultaneously or in an interleaved manner.

20. The apparatus of claim 17 wherein at least one of:
a) the streams have a lateral dimension or diameter of D and the stream-to-stream spacing or gap at the patient impact surface is between 0.5D and 100D;
b) two or more impacting streams produce a wetting meniscus which bridges across a span or gap between two said adjacent impact locations;
c) the apparatus is scanned relative to the patient or vice versa;
d) the patient is cooperatively or supportively treated with a drug, medicament or diet; and
e) one or both of the streamed liquid or a patient's body portion is controlled or monitored with respect to temperature.
